(19) United States
(12) Patent Application Publication
(10) Pub. No.: US 2024/0293264 A1
(43) Pub. Date: Sep. 5, 2024

(54) FLUID DRAINAGE AND DELIVERY DEVICE FOR WOUND TREATMENT

(71) Applicant: AROA BIOSURGERY LIMITED, Auckland (NZ)

(72) Inventors: Brian Roderick WARD, Pukekohe (NZ); Isaac Tristram MASON, Auckland (NZ); Hamish Rose, Auckland (NZ); Alister Todd JOWSEY, Auckland (NZ); Michael LOVELAND, Auckland (NZ); Liam DOUGLAS, Auckland (NZ); Samuel TURNER, Auckland (NZ); Brendan Andrew O'NEILL, Auckland (NZ)

(21) Appl. No.: 18/575,184

(22) PCT Filed: Jul. 4, 2022
(86) PCT No.: PCT/NZ2022/050090

§ 371 Date: Dec. 28, 2023

Related U.S. Application Data
(60) Provisional application No. 63/217,889, filed on Jul. 2, 2021.
(60) Provisional application No. 63/217,941, filed on Jul. 2, 2021.
(60) Provisional application No. 63/279,915, filed on Nov. 16, 2021.
(60) Provisional application No. 63/281,363, filed on Nov. 19, 2021.

(51) Int. Cl. A61F 13/05; A61M 1/00; A61M 39/10

(52) U.S. Cl. CPC A61F 13/05; A61M 1/85; A61M 1/913; A61M 1/92; A61M 2039/1061; A61M 2205/04

(57) ABSTRACT

A device for implantation at a treatment site in the body of a patient for the removal of fluid from the treatment site has a removable conduit structure, a connector, and a porous bioresorbable sheath. The removable conduit structure has two or more pieces and at least in part defines a fluid removal lumen, and the porous bioresorbable sheath surrounds at least a portion of the conduit structure. The connector removably and coaxially couples a first piece of the conduit structure to a second piece of the conduit structure and is configured to decouple from one or both of the conduit pieces upon initiation of removal of the conduit structure from the treatment site upon completion of treatment.

### TECHNICAL FIELD

The invention relates to a device for implanting at a wound treatment site, for the delivery of fluid to the site and for the drainage of fluid from the site. In particular, the device has a fluid supply lumen and a fluid removal lumen, and a bioresorbable sheath.

### BACKGROUND OF THE INVENTION

The drainage of fluid and the reduction of dead space from surgical or traumatic wounds is often a critical factor in the timely and effective recovery of a patient. Seromas and hematomas are pockets of serous fluid or blood that accumulate at wound sites post-surgery that can hinder recovery. In the absence of adequate drainage and dead space closure, poor healing, infection or dehiscence may lead to a requirement for additional surgery and longer hospital stays. Seromas and hematomas are common after reconstructive plastic surgery procedures, trauma, mastectomy, tumour excision, caesarean, hernia repair and open surgical procedures involving a lot of tissue elevation and separation.

While reducing dead space and providing drainage of fluid from a wound site is highly desirable in many instances, it is useful in other circumstances to be able to deliver fluid directly to a wound site to aid in the wound healing process. For example, instilling antimicrobial solutions locally to prevent infections. Similarly, instillation of local anaesthetics can aid pain management.

Prior art fluid removal devices are prone to blocking and are ineffective at preventing the formation of seroma within a soft tissue cavity. Loose tissue debris remaining at the site following surgery, such as loose connective tissue and adipose (fat) tissue, in combination with various biological factors such as fibrinogen and lysed cells tend to cause these devices to be substantially or completely block during use. Blockages reduce the ability of any device to remove fluid from a closed surgical wound and limit the effective delivery of vacuum pressure to a treatment site.

As a consequence, prior art fluid removal devices generally only apply a low level of suction (typically less than 60 mm Hg of vacuum). Further, attempting to operate these devices at higher vacuums does not improve their effectiveness, it simply hastens the speed at which the devices block.

It is therefore an object of the invention to provide a fluid drainage or delivery device that addresses one or more of the abovementioned shortcomings, and/or at least to provide a useful alternative to existing devices.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

### SUMMARY OF THE INVENTION

In a first aspect, the present invention provides a device for implantation at a treatment site in the body of a patient for the removal of fluid from the treatment site. The device comprises: a conduit structure at least in part defining a fluid removal lumen, the conduit structure comprising a first removable piece and a second removable piece; a connector removably and coaxially coupling the first piece of the conduit structure to the second piece of the conduit structure; and a porous bioresorbable sheath surrounding at least a portion of the conduit structure. The connector is configured to decouple from one or both of the conduit pieces upon initiation of removal of the conduit structure or a part thereof from the treatment site at the completion of treatment.

The device may comprise an inlet port and an outlet port and the conduit structure extends between the inlet port and the outlet port.

In an embodiment, the device has a dual lumen port for connection with one or more external components, wherein a first lumen of the port is connected to a first piece of the conduit structure and a second lumen of the port is connected to a second piece of the conduit structure.

In an embodiment, the connector is at a position on the device distal to the port(s).

In an embodiment, the two pieces of the conduit structure have abutted ends.

In an embodiment, the two pieces of the conduit structure are substantially the same length. Alternatively they may have different lengths.

In an embodiment, the connector is tubular.

In an embodiment, a first end of the connector is configured to fit snuggly in a lumen of the first part of the conduit structure and a second end of the connector is configured to fit snuggly in a lumen of the first part of the conduit structure.

In an embodiment, the connector is a bioresorbable component.

In an embodiment, the connector comprises a bioresorbable resilient truss having one or more flexible elongate wall members wound in a manner to define a channel, and one or more bracing members linked to the elongate wall member(s).

In an embodiment, two flexible elongate wall members wound in a manner to define a channel, the two elongate wall members intersecting each other periodically at a plurality of cross-over nodes.

In an embodiment, the two wall members are oppositely wound.

In an embodiment, a first one of the wall members is a left-side wall member, and a second one of the wall members is a right-side wall member.

The device may comprise at least two flexible elongate bracing members, each bracing member being linked to the two elongate wall members at a plurality of the cross-over nodes. The bracing members may be provided along the top and bottom of the channel.

In an embodiment, each bracing member is mechanically linked to the two elongate wall members at the respective cross-over nodes by way of the respective bracing member looping around the wall members.

In an embodiment, the connector is attached to the sheath.

In an embodiment, the bioresorbable sheath comprises a plurality of apertures positioned to enable fluid communication between the treatment site and the conduit structure, the apertures each having an area of about 1 mm² or less.

In an embodiment, the sheath comprises one or more top sheets that extends over a top part of the conduit structure, and one or more bottom sheets that extend over a bottom part of the conduit structure.

In an embodiment, the top and bottom sheets are stitched together. The connector may be tied to a row of stitching. Alternatively, the connector may be otherwise secured to the sheath.

In an embodiment, the top and bottom sheets are mechanically interlocked together.

In an embodiment, the sheath comprises a first sheet having a plurality of lugs and a second sheet having a plurality of apertures, each lug of the first sheet being located through a respective aperture in the second sheet to interlock the first sheet with the second sheet.

In an embodiment, the top sheet comprises a plurality of lugs and the underlying sheet(s) comprise(s) a plurality of apertures, each lug of the top sheet being located through a respective aperture in the underlying sheet(s) to interlock the sheets of the sheath.

In an embodiment, the holes and the lugs are dimensioned so that the lugs engage with a surface of the second sheet.

In an embodiment, the sheath comprises an end section proximal an inlet and outlet of the device, configured to prevent or minimise the ingress of wound debris into the conduit structure.

In an embodiment, the end section of the sheath does not comprise through apertures.

In an embodiment, the sheath comprises one or more layers of extracellular matrix (ECM) or polymeric material. The ECM may be formed from decellularised propria-submucosa of a ruminant forestomach.

In an embodiment, the removable conduit structure comprises a silicone form.

In an embodiment, the fluid removal lumen has a cross-sectional area of at least 7 mm². For example, the fluid removal lumen may have a cross-sectional area of about 18 mm².

In an embodiment, the sheath comprises a sealing end section free from apertures and having a tight fit with the underlying portion of the conduit structure.

In an embodiment, the sealing end section of the sheath extends over a portion of the conduit structure that comprises fluid impervious walls.

In an embodiment the connector is an elongate component that forms a portion of the conduit structure and which defines a respective portion of the fluid removal lumen.

In a second aspect, the present invention provides system for draining fluid from a treatment site and delivering fluid to a treatment site in the body of a patient comprising:
(i) a device according to the first aspect;
(ii) a conduit releasably coupled to either a port of the device or to a fluid impermeable dressing;
(iii) a reservoir located external to the body of the patient and containing a treatment fluid, the reservoir in fluid communication with the fluid supply lumen;
(iv) a second reservoir located external to the body of the patient, the second reservoir in fluid communication with fluid removal lumen for receiving fluid from the device; and
(v) a source of pressure coupled to the conduit for delivering positive pressure or negative pressure to the device.

In an embodiment, the source of pressure is capable of delivering negative pressure to the device so that fluid is drained from the treatment site into the device and transferred through the conduit to the reservoir.

In an embodiment, the port of the device is positioned external to the patient's body.

In a second aspect, the present invention provides a kit of parts for forming the device of the first aspect, comprising a two-piece conduit structure defining a fluid removal lumen, a connector for removably and coaxially coupling a first piece of the conduit structure to a second piece of the conduit structure; and a bioresorbable sheath defining a passage for receipt of the conduit structure.

In an embodiment, the bioresorbable sheath is generally tubular having two open ends.

Also described herein is a device for implantation at a treatment site in the body of a patient for the removal of fluid from the treatment site. The device comprises a conduit structure at least in part defining a fluid removal lumen, and a porous bioresorbable sheath surrounding a portion of the conduit structure. The conduit structure comprises a removable component configured for removal from the treatment site upon completion of treatment.

In an embodiment, the device is configured to deliver a fluid to the treatment site, and wherein the conduit structure further defines a fluid supply lumen. One end of the fluid supply lumen may be in fluid communication with one end of the fluid removal lumen.

In an embodiment, the device comprises a dual lumen port for connection with one or more external components, wherein a first lumen of the port is in fluid communication with the fluid removal lumen.

In an embodiment, the bioresorbable sheath comprises a plurality of apertures positioned to enable fluid communication between the treatment site and the conduit structure, the apertures each having an area of about 1 mm² or less. For example, the apertures in the sheath may each have an area of between about 0.2 mm² to about 0.8 mm².

In an embodiment, the sheath comprises a top sheet that wraps over a top part of the conduit structure, and a bottom sheet that wraps over a bottom part of the conduit structure, wherein the top and bottom sheets are joined around the conduit structure along a side seam. The top and bottom sheets may be stitched together, for example with a bioresorbable suture.

In an embodiment, the sheath forms one or more flange(s) or tab(s) extending beyond the side seam, for securing the device to tissue at the treatment site. The flanges or tabs may comprise two layers, and the layers are attached at or near an edge of the flange or tab.

In an embodiment, the apertures in the sheath are provided on upper and lower surfaces of the device.

In an embodiment, the sheath comprises an end section proximal an inlet and outlet of the device, configured to prevent or minimise the ingress of wound debris into the conduit structure. The end section of the sheath preferably does not comprise through apertures.

In an embodiment, an end of the sheath distal an inlet and outlet of the device is closed.

Alternatively, an end of the sheath distal an inlet and outlet of the device may be open. In an embodiment, the sheath comprises one or more layers of extracellular matrix (ECM) or polymeric material. The ECM may be formed from decellularised propria-submucosa of a ruminant forestomach.

In an embodiment, the fluid supply lumen of the removable conduit structure comprises a non-porous wall along at least a major part of the length of the structure.

The fluid removal lumen of the removable conduit structure may comprise a porous wall along a major part of the length of the structure.

In an embodiment, the removable conduit structure comprises a truss defining at least a major portion of the fluid removal lumen of the removable conduit structure. In an embodiment, the truss comprises two flexible elongate wall members wound such that they intersect each other periodically at a plurality of cross-over nodes. Each elongate wall member may be generally helical, and wherein the two wall members are oppositely wound. The truss may form a flexible tube having a round or oval cross-section.

In some embodiment trusses, the truss may include at least two flexible elongate bracing members, each bracing member being linked to the two elongate wall members at a plurality of the cross-over nodes. The bracing members may extend generally longitudinally along a side of the channel. The bracing truss members may be provided on opposite sides of the channel. Each bracing member may be bonded to the two elongate wall members at the respective cross-over nodes.

In an embodiment, the truss may include a securing truss member, wound to secure the truss of the fluid removal lumen to the fluid supply lumen.

In an embodiment, the removable conduit structure comprises a silicone form.

In an embodiment the fluid removal lumen has a cross-sectional area of at least 7 mm², for example a cross-sectional area of about 18 mm².

In an embodiment, the fluid removal lumen has an inlet end and an outlet end, and wherein the fluid supply lumen is configured to supply fluid to adjacent the inlet end of the fluid removal lumen.

In an embodiment, the fluid supply lumen and the fluid removal lumen are generally the same length and positioned adjacent each other.

In an embodiment, the fluid supply lumen and the fluid removal lumen are colinear. For example, the device may form a loop. In one embodiment, the loop comprises two limbs of the conduit structure with abutted ends.

In an embodiment, the device comprises a port in fluid communication with the fluid removal and/or fluid supply lumens and being connectable to a source of negative pressure or positive pressure.

The treatment site may be a region between surfaces or planes of muscle tissue, connective tissue and/or or skin tissue that have been separated during surgery or as a result of trauma, or a region within a layer of tissue.

In an embodiment, the sheath comprises a sealing end section free from apertures and having a tight fit with the underlying portion of the conduit structure. The sealing end section of the sheath extends over a portion of the conduit structure that comprises fluid impervious walls.

In an embodiment, the cross-sectional area of the sheath and the underlying conduit structure is reduced along at least a portion of the sealing section.

In an embodiment, the cross-sectional area of the sheath and the underlying conduit structure is tapered along at least a portion of the sealing section.

Also described herein is a device for implantation at a treatment site in the body of a patient for the delivery of fluid to and/or removal of fluid from the treatment site. The device comprises: a conduit structure defining a fluid supply and/or removal lumen and a bioresorbable sheath surrounding a portion of the removable conduit structure. The sheath comprises a plurality of apertures sized and positioned to enable fluid communication between the treatment site and the conduit structure while preventing blockages in the device.

In an embodiment, the apertures in the sheath each have an area of between about 0.2 mm² to about 0.8 mm².

In an embodiment, the sheath comprises a sealing end section free from apertures and having a tight fit with the underlying portion of the conduit structure.

In an embodiment, the sealing end section of the sheath extends over a portion of the conduit structure that comprises fluid impervious walls.

In an embodiment, the cross-sectional area of the sheath and the underlying conduit structure is reduced along at least a portion of the sealing section.

In an embodiment, the cross-sectional area of the sheath and the underlying conduit structure is tapered along at least a portion of the sealing section.

In an embodiment, the device comprises a port in fluid communication with the lumen(s) of the conduit structure.

In an embodiment, the conduit structure comprises a removable component configured for removal from the treatment site upon completion of treatment.

The device according to the second aspect may include any one or more of the features described above in relation to the first aspect.

Also described herein is a device for implantation at a treatment site in the body of a patient for the delivery of fluid to and/or removal of fluid from the treatment site; the device comprising:
a conduit structure defining a fluid supply lumen and a porous fluid removal lumen, one end of the fluid supply lumen being in fluid communication with a first end of the fluid removal lumen;
a bioresorbable sheath surrounding a portion of the removable conduit structure; and
a port in fluid communication with the fluid supply lumen and/or the fluid removal lumen(s).

In an embodiment, the device comprises a dual lumen port, with a first lumen of the port in fluid communication with the fluid supply lumen and a second lumen of the port in fluid communication with the fluid removal lumen.

A portion of the conduit structure defining the fluid supply lumen may be integrally formed with a portion of the conduit structure defining the fluid removal lumen.

In an embodiment, the fluid supply lumen and fluid removal lumen are co-axial. Alternatively, the fluid supply lumen and fluid removal lumen may be substantially parallel.

In an embodiment, the port is configured for connection with one or more external components.

In an embodiment, the sheath comprises a multiplicity of apertures to facilitate fluid transfer across the sheath, each aperture having an area of between about 0.2 mm² to about 0.8 mm².

In an embodiment, the sheath comprises a sealing end section free from apertures and having a tight fit with the underlying portion of the conduit structure.

In an embodiment, the conduit structure comprises a removable component configured for removal from the treatment site upon completion of treatment.

The device according to the third aspect may include any one or more of the features described above in relation to the first or second aspects.

Also described herein is a system for draining fluid from a treatment site and delivering fluid to a treatment site in the body of a patient comprising:
(vi) a device as described above;
(vii) a conduit releasably coupled to either a port of the device or to a fluid impermeable dressing;
(viii) a reservoir located external to the body of the patient and containing a treatment fluid, the reservoir in fluid communication with the fluid supply lumen;
(ix) a second reservoir located external to the body of the patient, the second reservoir in fluid communication with fluid removal lumen for receiving fluid from the device; and
(x) a source of pressure coupled to the conduit for delivering positive pressure or negative pressure to the device.

In an embodiment, the source of pressure is capable of delivering negative pressure to the device so that fluid is drained from the treatment site into the device and transferred through the conduit to the reservoir.

In an embodiment, the port of the device is positioned external to the patient's body.

Also described herein is a kit of parts for forming the device as described above, comprising a conduit structure defining a fluid removal lumen, and a bioresorbable sheath defining a passage for receipt of the conduit structure.

In an embodiment, the bioresorbable sheath is generally tubular having two open ends.

This invention may also be said broadly to consist in the parts, elements and features referred to or indicated in the specification of the application, individually or collectively, and any or all combinations of any two or more said parts, elements or features. Where specific integers are mentioned herein which have known equivalents in the art to which this invention relates, such known equivalents are deemed to be incorporated herein as if individually described.

The term ‘comprising’ as used in this specification and claims means ‘consisting at least in part of’. When interpreting statements in this specification and claims that include the term ‘comprising’, other features besides those prefaced by this term can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range and any range of rational numbers within that range (for example, 1 to 6, 1.5 to 5.5 and 3.1 to 10). Therefore, all sub-ranges of all ranges expressly disclosed herein are hereby expressly disclosed.

As used herein the term ‘(s)’ following a noun means the plural and/or singular form of that noun. As used herein the term ‘and/or’ means ‘and’ or ‘or’, or where the context allows, both.

### BRIEF DESCRIPTION OF THE FIGURES

The present invention will now be described by way of example only and with reference to the accompanying drawings in which:

FIG. 1 is a right-top perspective view showing a first embodiment device having securing tabs;

FIG. 2 is a left-underside side perspective view showing the device of FIG. 1;

FIG. 3 is a top detail view of the device in FIGS. 1 and 2;

FIG. 4 is a side elevation detail view of the embodiment of FIGS. 1 to 3;

FIG. 5 is a detail perspective view corresponding to FIG. 1;

FIG. 6 is a detail perspective view corresponding to FIG. 2;

FIG. 7 is perspective view of an exemplary conduit structure having a truss form;

FIG. 8 is a further perspective view of the conduit structure of FIG. 7;

FIG. 9 is a side detail view of the conduit structure of FIGS. 7 and 8;

FIG. 10 is a side detail view of the truss from the conduit structure of FIGS. 7 to 9;

FIG. 11A is a section view through the truss of FIG. 10, taken through the porous portion of the fluid removal lumen;

FIG. 11B is an end view of the integrally formed portion of the conduit structure of FIGS. 7 to 10 showing the device inlet and outlet;

FIG. 11C is a section view through the conduit structure of FIGS. 7 to 10, taken through the porous portion of the fluid removal lumen;

FIG. 12 is a perspective view of the truss from the conduit structure of FIGS. 7 to 10;

FIG. 13 is a top, cut-away view illustrating of an alternative embodiment device having a truss-type conduit structure;

FIG. 14 is a cut-away perspective corresponding to FIG. 13;

FIG. 15 is a cut-away detail perspective view of the embodiment of FIGS. 13 and 14;

FIG. 16 is a right-top perspective view showing a further embodiment device having a continuous securing flange;

FIG. 17 is a left-underside side perspective view showing the device of FIG. 16;

FIG. 18 is a top detail view of the device in FIGS. 16 and 17;

FIG. 19 is a side elevation detail view of the embodiment of FIGS. 16 to 18;

FIG. 20 is an end view of the embodiment of FIGS. 16 to 19;

FIG. 21 is a section view of through the conduit structure of the device of FIGS. 16 to 20;

FIGS. 22A and 22B illustrate and alternative embodiment conduit structure, where FIG. 22A is a partial perspective section view, and FIG. 22B is a partial top detail view;

FIG. 23 is a cut-away perspective view showing a further embodiment device that is adjustable to shorten the length of the device;

FIG. 24 is a perspective view of the dual lumen conduit of the device of FIG. 23;

FIGS. 25(i) to 25(vi) are partial top views illustrating the process of shortening the device of FIG. 23, where FIG. 25(i) illustrates cutting the device, FIG. 25(ii) shows the cut end of the device, FIG. 25(iii) is a cut-away view showing the cut device, FIG. 25(iv) is a cut-away view showing the conduit structure adjusted to its new position, FIG. 25(v) illustrates flattening the end of the device, and FIG. 25(vi) shows the end folded under to form a seal;

FIG. 26 is a perspective view of one end of an alternative embodiment conduit structure;

FIG. 27 is a top cut-away section view of the conduit structure of FIG. 26;

FIG. 28 is a perspective view of one end of an alternative embodiment conduit structure;

FIG. 29 is a top cut-away section view of the conduit structure of FIG. 28;

FIG. 30 is a perspective view of one end of an alternative embodiment conduit structure;

FIG. 31 is a top cut-away section view of the conduit structure of FIG. 30;

FIG. 32 is a perspective view of one end of an alternative embodiment conduit structure;

FIG. 33 is a top cut-away section view of the conduit structure of FIG. 32;

FIG. 34 is a perspective view of one end of an alternative embodiment conduit structure;

FIG. 35 is a top cut-away section view of the conduit structure of FIG. 34;

FIG. 36 is a perspective view showing a fourth embodiment device with a loop structure;

FIG. 37 is a further perspective view of the embodiment of FIG. 36;

FIG. 38 is a top view of the embodiment of FIGS. 36 and 37;

FIG. 39 is a top cut-away view of the embodiment of FIGS. 36 and 37;

FIG. 40 is a section view of the fluid removal lumen in the embodiment of FIGS. 36 to 39;

FIG. 41 is a cut-away perspective view showing a further embodiment device with a loop structure;

FIG. 42 is a section view taken through the fluid removal lumen of the device of FIG. 41;

FIG. 43 is a plan view of the conduit structure of the device of FIG. 41, illustrating the direction of flow through the device;

FIG. 44 is an end view of the conduit structure of FIG. 43;

FIG. 45 is a plan view of the conduit structure of FIGS. 43 and 44, before assembly within the sheath;

FIG. 46 is a cut-away perspective view showing a further embodiment device with a loop structure;

FIG. 47 is a cut-away plan view corresponding to the embodiment shown in FIG. 46;

FIG. 48 is a section view taken through the fluid removal lumen of the device of FIGS. 46 and 47;

FIG. 49 is a plan view of the conduit structure of the device of FIGS. 46 to 48, illustrating the direction of flow through the device;

FIG. 50 is an end view of the conduit structure of FIG. 49;

FIG. 51 is a plan view of the conduit structure of FIGS. 49 and 50, before assembly within the sheath;

FIG. 52 is a top perspective view showing a fifth embodiment device with a loop structure;

FIG. 53 is an underside perspective view of the device of FIG. 52;

FIG. 54 is a section view of the fluid removal lumen in the embodiment of FIGS. 52 and 53;

FIG. 55 is a top perspective view showing a further embodiment device with a loop structure;

FIG. 56 is a cut-away perspective view of the device of FIG. 55;

FIG. 57 is a top perspective view showing a further embodiment device with a loop structure;

FIG. 58 is a top perspective view showing a further embodiment device with a loop structure with some of the top sheath cut-away;

FIG. 59 is a top perspective view showing a further embodiment device having a loop configuration, held in a multilayer structure;

FIG. 60 is a section view of a portion of the device of FIG. 59, taken through the conduit structure of FIG. 59; and

FIG. 61 is a cut-away top perspective view of a further embodiment device with a loop structure formed from two limbs with a truss-based connector;

FIG. 62 is a cut-away detail view of the connection between the two limbs in the embodiment of FIG. 61;

FIG. 63 is a section view taken through line XX of FIG. 62;

FIG. 64 is the detail view of FIG. 62, but with end portions of the two limbs of the conduit structure shown cut away;

FIG. 65 is the detail view of FIG. 62, but with the conduit structure hidden to show the truss-based connector;

FIG. 66 is a cut-away top perspective view of a further embodiment device with a loop structure, in which the connector forms a portion of the conduit structure;

FIGS. 67A and 67B illustrate directional fluid flow through a sheet of ECM, where FIG. 67A illustrates an exemplary ECM structure prior to processing, and FIG. 67B illustrates the structure following processing and the directional bias of fluid flow through the ECM sheet.

### DETAILED DESCRIPTION

### Definitions

The term “bioresorbable” as used herein means able to be broken down and absorbed or remodelled by the body, and therefore does not need to be removed manually.

The term “treatment site” as used herein refers to a site in a human or animal body where surfaces of muscle tissue, connective tissue or skin tissue have been separated during surgery or as a result of trauma or removal.

The term “propria-submucosa” as used herein refers to the tissue structure formed by the blending of the lamina propria and submucosa in the forestomach of a ruminant.

The term “lamina propria” as used herein refers to the luminal portion of the propria-submucosa, which includes a dense layer of extracellular matrix.

The term “extracellular matrix” (ECM) as used herein refers to animal or human tissue that has been decellularised and provides a matrix for structural integrity and a framework for carrying other materials.

The term “decellularised” as used herein refers to the removal of cells and their related debris from a portion of a tissue or organ, for example, from ECM.

The term “helical” as used herein refers to a generally spiralling form, it may relate to a form with a circular cross-section, but also refers to forms with non-circular cross sections.

The term “polymeric material” as used herein refers to large molecules or macromolecules comprising many repeated subunits, and may be natural materials including, but not limited to, polypeptides and proteins (e.g. collagen), polysaccharides (e.g. alginate) and other biopolymers such as glycoproteins, or may be synthetic bioresorbable materials including, but not limited to polyglycolic acid, polylactic acid, P4HB (Poly-4-hydroxybutyrate), polylactic and polyglycolic acid copolymers, polycaprolactone, polydioxanone and poly(trimethylene carbonate) or they may be non-absorbable materials such polypropylene, polyester, polytetrafluoroethylene, polyamide and polyethylene.

### Device

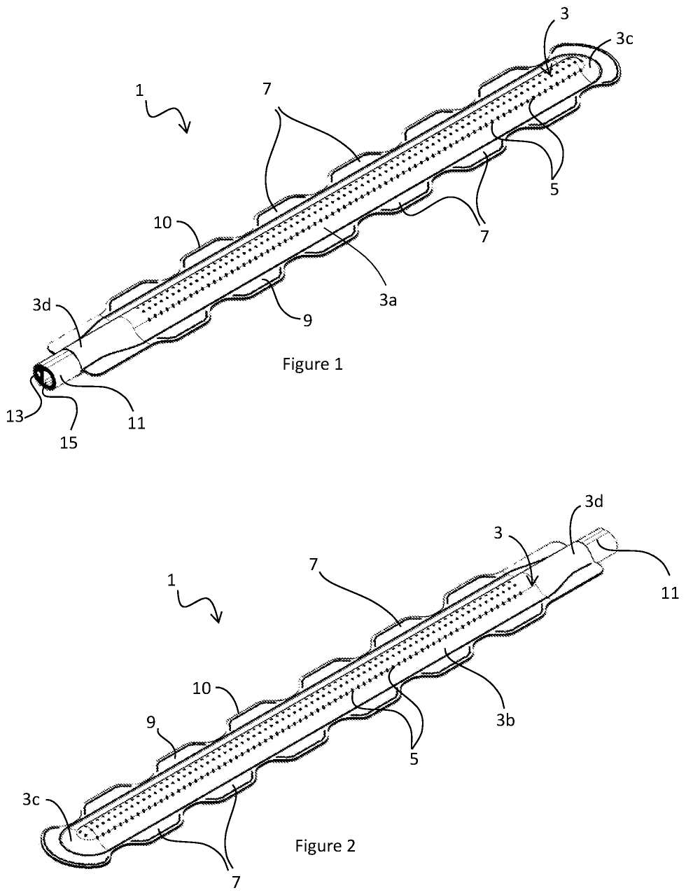

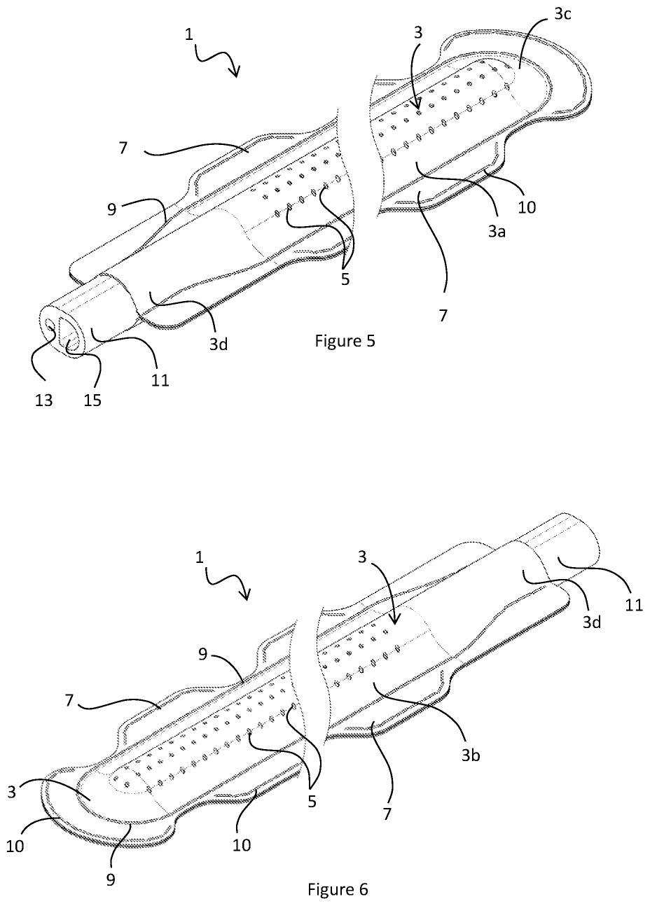

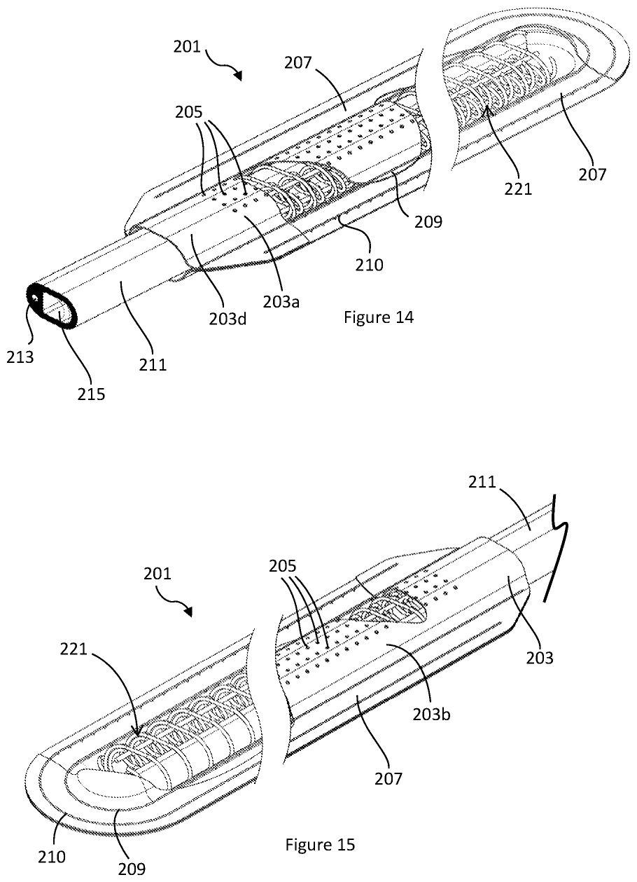

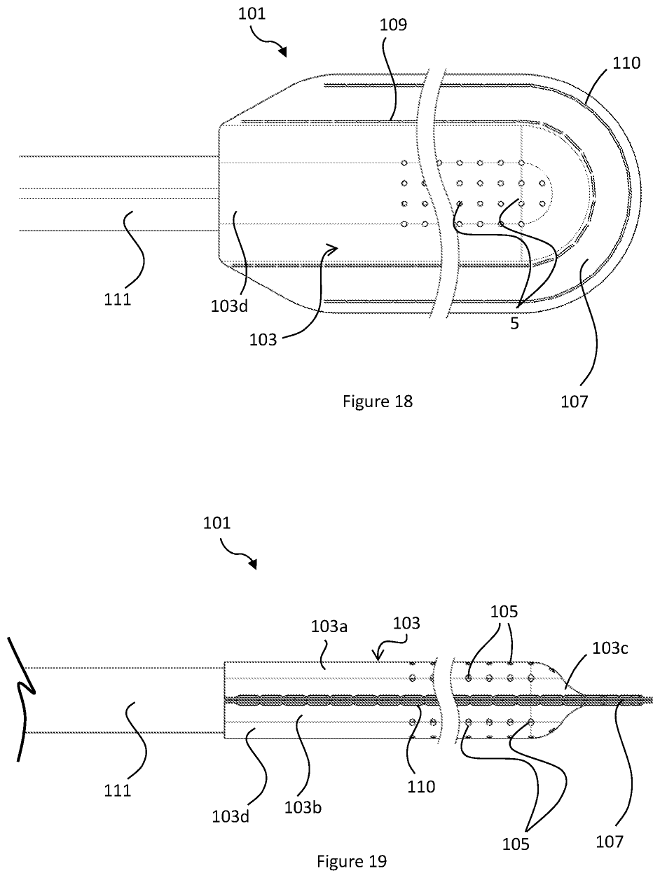

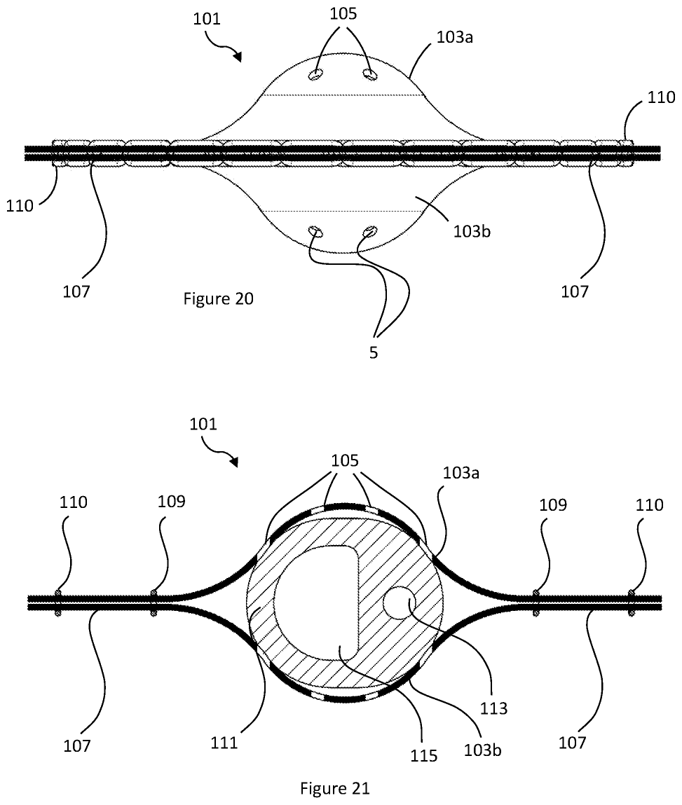

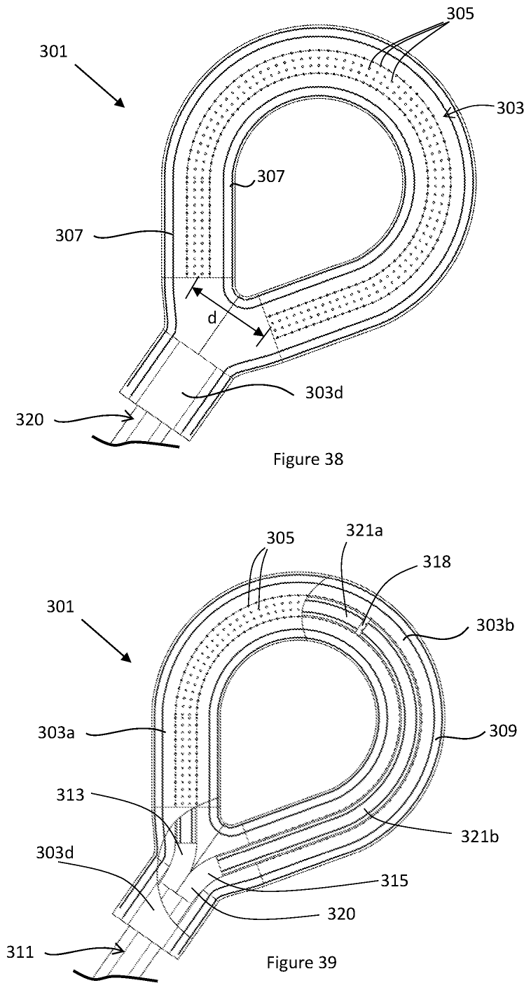

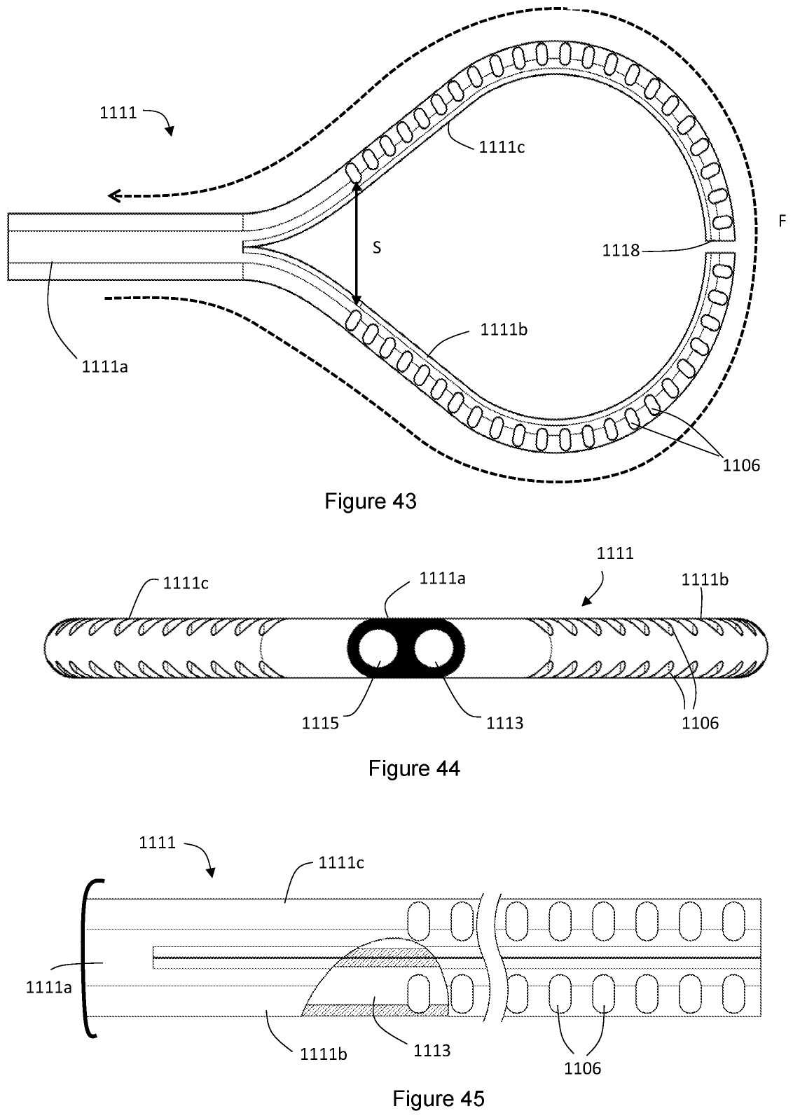

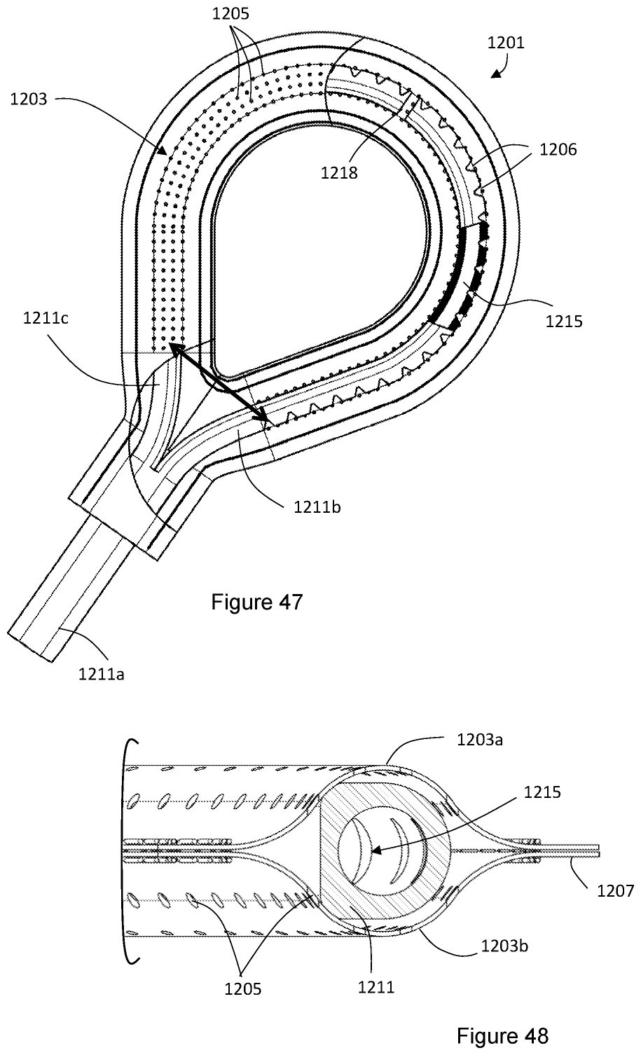

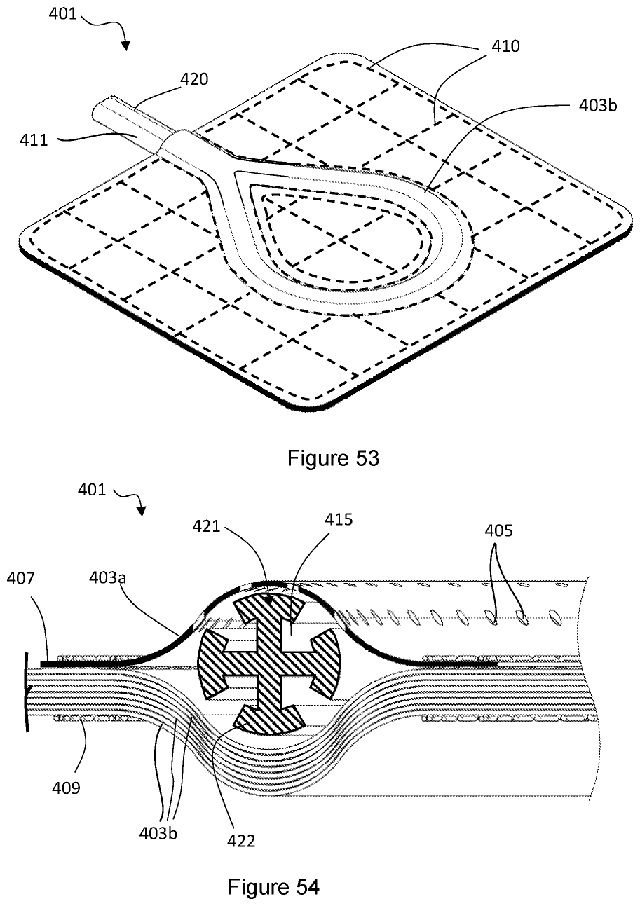

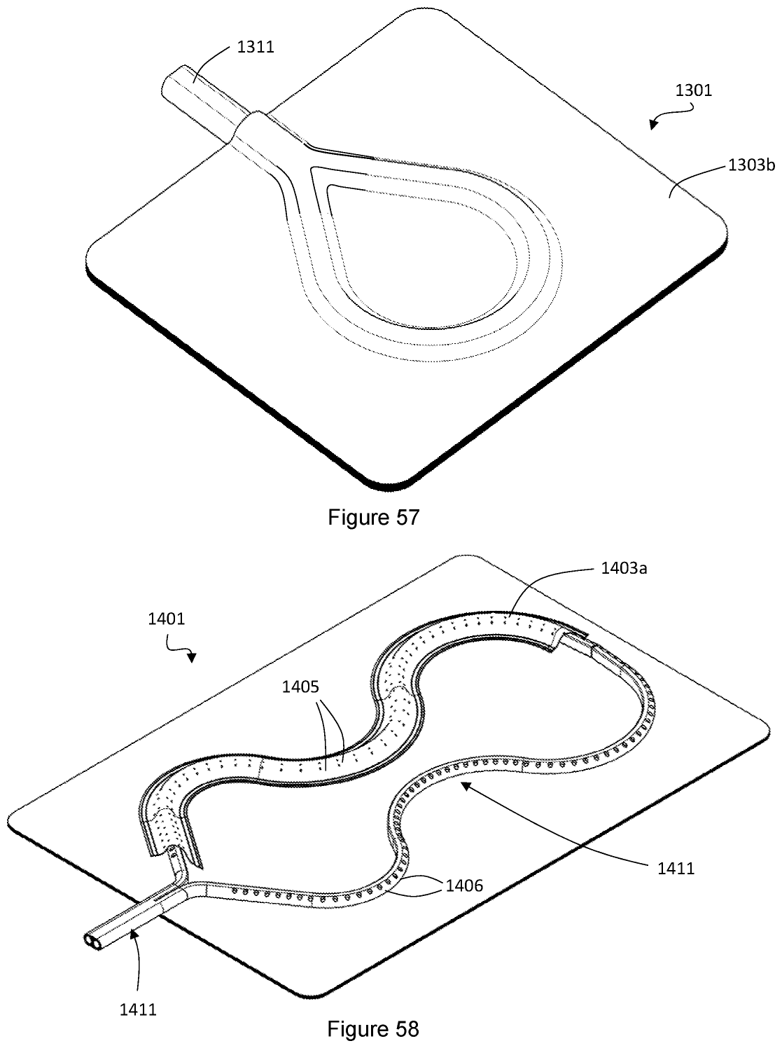

Various embodiments of the device and system of the present invention will now be described with reference to FIGS. 1 to 59B. In these figures, unless otherwise described, like reference numbers are used to indicate like features. Where various embodiments are illustrated, like reference numbers may be used for like or similar features in subsequent embodiments but with the addition of a multiple of 100, for example 2, 102, 202, 302 etc.

Directional terminology used in the following description is for ease of description and reference only, it is not intended to be limiting. For example, the terms ‘front’, ‘rear’, ‘upper’, ‘lower’, and other related terms are generally used with reference to the way the device is illustrated in the drawings.

FIG. 1 illustrates one embodiment device 1 for implantation at a treatment site in the body of a patient for delivering fluid to the treatment site and also for draining fluid from the treatment site. The drained fluid may include the treatment fluid and/or wound exudate. The device comprises a bioresorbable porous sheath 3 that surrounds a removable conduit structure 11. The conduit structure 11 acts to hold apart two tissue surfaces of the wound treatment site to create a channel for delivering and removing fluid.

The device 1 is a flexible device such that the device can generally conform to the contours of a wound site. The device may be elongate, but may have other forms.

The conduit structure 11 is a flexible structure comprising a material that is non-resorbable by a body, such that the conduit structure is configured to be removed at the end of the treatment. The conduit structure defines a fluid supply lumen 13 and a fluid removal lumen 15. The fluid supply lumen and fluid removal lumen may be positioned side-by-side or may be coaxial.

The fluid supply lumen 13 is a generally closed wall lumen configured to supply a fluid to an inlet end of the fluid removal lumen. In contrast, the fluid removal lumen 15 has a generally porous wall along a length of the lumen, to allow fluid communication between the fluid removal conduit and the treatment area. The fluid removal lumen may have a circular or non-circular cross section. The fluid removal lumen has a cross-sectional area of at least 16 mm², for example an area of 18 mm².

The bioresorbable sheath 3 surrounding the conduit structure comprises a plurality of apertures 5 positioned to enable fluid communication across the sheath 3, between the treatment site and the conduit structure. The apertures 5 each have an area of about 1 mm² or less, preferably about 0.8 mm² or less, for example between about 0.2 mm² and about 0.5 mm². If the apertures are too small, the device 1 may be ineffective for prevention of seroma formation. If the apertures 5 are too large, wound debris such as fatty tissue may be drawn into the device and cause blockages.

The fit of the sheath 3 over the conduit structure 11 should be tight to ensure the sheath isn't sucked into the fluid removal lumen on the application of negative pressure, and to minimise the likelihood of wound debris entering the conduit structure other than through the sheath apertures 5. In preferred embodiments, the sheath 3 comprises top and bottom sheets 3a, 3b that wrap over and sandwich the conduit structure 11 between the sheets. The top and bottom sheets 3a 3b are joined together along a side seam 9, along the side of the conduit structure 11, the side seam 9 may comprise one or more rows of stitching, for example. The stitching may be resorbable, for example comprising a bioresorbable suture.

The sheath 3 comprises one or more flange, or tabs 7 for securing the device 1 to the wound treatment site, for example by suturing the flange or tab 7 to tissue at the wound treatment site. This ability to secure the device enables accurate placement of the device 1 at the wound site, and reduces the likelihood of the device moving away from the installed position, particularly for treatment sites that undergo high levels of movement. Securing the sheath 3 of the device 1 to tissue at the treatment site also allows the removable conduit structure 11 to be removed while minimising movement of the sheath 3, and thereby reduces disruption to surrounding tissue which may have bonded with the sheath 3.

The flange or tabs 7 extend out beyond the side seam 9, and preferably comprise both the top and bottom sheath layers 3a, 3b to provide a stronger connection with the securing sutures and to stiffen the flange or tabs to improve the ease of stitching. The flange or tabs 7 may be stitched together at or near and edge of the flange of tab 7 along a peripheral stitch line 10 to prevent the sheets 3a, 3b separating.

In some embodiments of the invention, the sheath 3 is formed from extracellular matrix (ECM). The ECM sheets are typically collagen-based biodegradable sheets comprising highly conserved collagens, glycoproteins, proteoglycans and glycosaminoglycans in their natural configuration and natural concentration. ECM can be obtained from various sources, for example, dermis pericardial or intestinal tissue harvested from animals raised for meat production, including pigs, cattle and sheep or other warm-blooded vertebrates.

The ECM tissue suitable for use in the invention comprises naturally associated ECM proteins, glycoproteins and other factors that are found naturally within the ECM depending upon the source of the ECM. One source of ECM tissue is the forestomach tissue of a warm-blooded vertebrate. The ECM suitable for use in the invention may be in the form of sheets of mesh or sponge.

Forestomach tissue is a preferred source of ECM tissue for use in this invention. Suitable forestomach ECM typically comprises the propria-submucosa of the forestomach of a ruminant. In particular embodiments of the invention, the propria-submucosa is from the rumen, the reticulum or the omasum of the forestomach. These tissue scaffolds typically have a contoured luminal surface. In one embodiment, the ECM tissue contains decellularised tissue, including portions of the epithelium, basement membrane or tunica muscularis, and combinations thereof. The tissue may also comprise one or more fibrillar proteins, including but not limited to collagen I, collagen Ill or elastin, and combinations thereof. These sheets are known to vary in thickness and in definition depending upon the source of vertebrate species.

The method of preparing ECM tissues for use in accordance with this invention is described in U.S. Pat. No. 8,415,159.

In some embodiments of the invention, sheets of polymeric material may be used. The polymeric material may be in the form of sheet or mesh. Synthetic materials such as polyglycolic acid, polylactic acid and poliglecaprone-25 will provide additional strength in the short-term, but will resorb in the long term. Alternatively, the polymeric material may be a natural material, or derived from a natural material, such as a proteins (e.g. collagen), a polysaccharides (e.g. alginate), and a glycoprotein (e.g. fibronectins).

Any desirable bioactive molecules can be incorporated into the ECM or polymeric material. Suitable molecules include for example, small molecules, peptides or proteins, or mixtures thereof. The bioactive materials may be endogenous to ECM or maybe materials that are incorporated into the ECM and/or polymeric material during or after the grafts manufacturing process. In some embodiments, two or more distinct bioactive molecules can be non-covalently incorporated into ECM or polymer. Bioactive molecules can be non-covalently incorporated into material either as suspensions, encapsulated particles, micro particles, and/or colloids, or as a mixture thereof. Bioactive molecules can be distributed between the layers of ECM/polymeric material. Bioactive materials can include, but are not limited to, proteins, growth factors, antimicrobials, and anti-inflammatoireluding doxycycline, tetracyclines, silver, FGF-2, TGF-B, TGF-B2, BMR7, BMP-12, PDGF, IGF, collagen, elastin, fibronectin, and hyaluronan.

FIGS. 1 to 6 show a first exemplary embodiment device 1 having a sheath 3 with an upper sheet 3a, and a lower sheet 3b. As described previously, the upper and lower sheets 3a, 3b are joined at a sewn seam 9 along the side of the conduit structure 11, with tabs 7 protruding from the seam. Apertures 5 are provided in both the top 3a and bottom 3b sheath sheets. Referring to FIG. 4, the apertures 5 are distributed such that some of the apertures 5 are positioned on a side of the device. These side apertures may be helpful in some applications to allow negative pressures to be continued to be applied to the treatment site if the top apertures are in contact with a tissue surface. In the embodiment shown, the two outer rows of apertures 5 on the upper sheet 3a open to an angle upwards and outwards, and the two outer rows of apertures 5 on the lower sheet 3b open to an angle downwards and outwards.

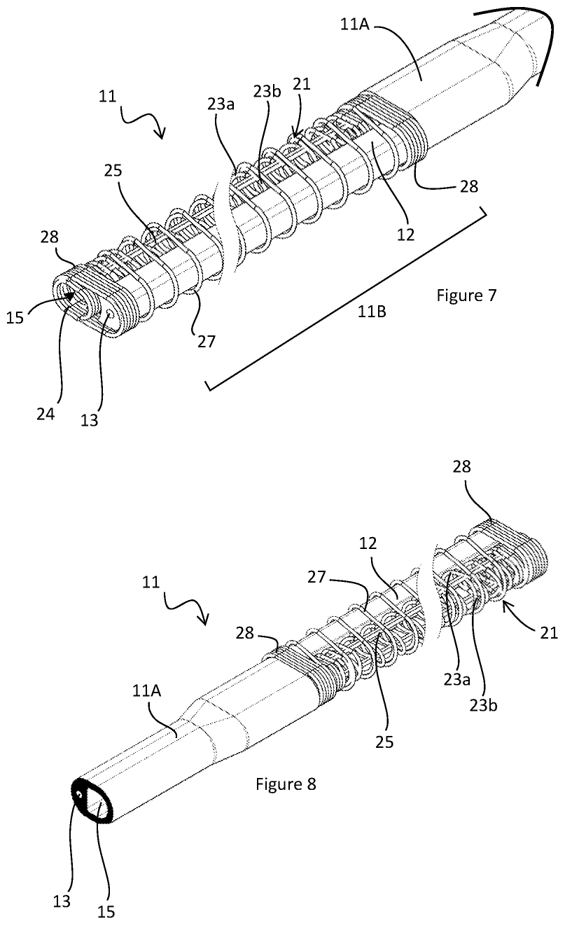

The device 1 has an elongate shape with both the inlet and outlet ports for the device provided at the same end, and with an opposite closed end 3c of the device. The sheath 3 comprises a sealing end section 3d at the end of the sheath proximal the inlet and outlet, where the conduit 11 protrudes from the sheath 3. This end section 3d is free from apertures and extends over a portion of the conduit structure 11A that comprises fluid impervious walls (Further illustrated in FIGS. 7 to 10). This end section 3d forms a tight fit with the underlying conduit structure 11 and acts to provide a type of seal with the conduit structure 11 that prevents or reduces the ingress of wound debris, tissue debris and fat between the sheath 3 and conduit structure 11, which has the potential to cause blockages.

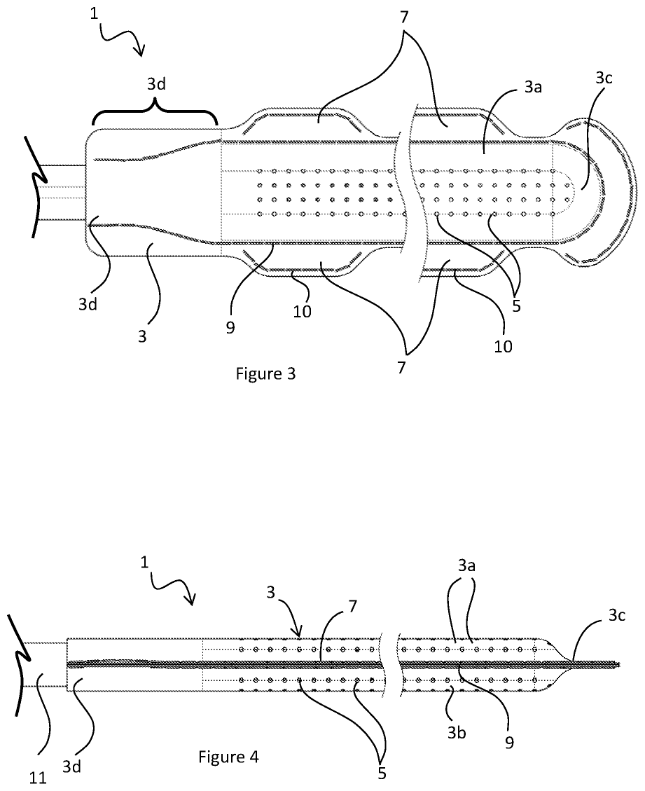

The conduit structure 11 and sheath 3 may neck at or along the sealing end section 3d of the sleeve 3 to create a smaller cross section at the opening of the sheath 3, as best illustrated in FIG. 3. This necked section further enhances the seal between the sheath 3 and the outer surface of the conduit structure 11 at the end region 3d and improves the retention of the conduit structure 11 within the sheath 3. This configuration is particularly intended for use in embodiments in which the conduit structure comprises a truss, as described in more detail below.

In some embodiments the seal between the sealing end section 3d and the conduit structure 11 could be further improved by lengthening this end section 3d. Additionally, or alternatively, the sheath 3 could be tied to the conduit structure 11 at this section 3d, using a noose-type tie, tightly wrapping around the sheath. In some embodiments, an additional sheet of bioresorbable material may be wrapped around the conduit 11 at this end section 3d to improve the seal.

In the embodiment shown in FIG. 1, the device 1 comprises a removable conduit structure 11 having the fluid supply lumen 13 and removal lumen 15 arranged side-by-side. In this embodiment, a length of the conduit structure 11 (including the length external of the sheath) consists of an extruded dual lumen conduit. The fluid supply lumen 13 is a generally closed wall tube with fluid impervious walls, that is positioned in the sheath 3 to deliver fluid to the distal, closed end 3c of the device 1 and thereby to the inlet end of the fluid removal lumen 15. The outlet end of the inlet lumen 13 is adjacent to and in fluid communication with the inlet end of the fluid removal lumen 15. Since no part of the fluid supply lumen 13 is in fluid communication in a downstream direction from the wound site, supplied fluid is delivered to the fluid removal lumen 15 consistently without blockages occurring in the supply lumen 13.

FIGS. 7 to 12 illustrate one exemplary embodiment conduit structure 11 for use in the embodiment of FIGS. 1 to 6. In this embodiment, the conduit structure comprises two portions, a first integrally formed portion 11A positioned proximal the first end of the sheath 3, and protruding from the sheath, and a second portion 11B wholly contained in the sheath 3.

The integrally formed portion 11A of the conduit structure 11 comprises a dual lumen conduit defining a first portion of the inlet lumen 13 and a second portion of the outlet lumen 15 and forming the inlet and outlet to the device 1. The lumens 13, 15 of the integrally formed portion 11A comprise impervious walls with no through apertures. Typically, the inlet lumen 13 for the fluid supply is significantly smaller than the larger fluid removal lumen 15. The integrally formed portion 11A may be a moulded piece and preferably formed from a material such as silicone.

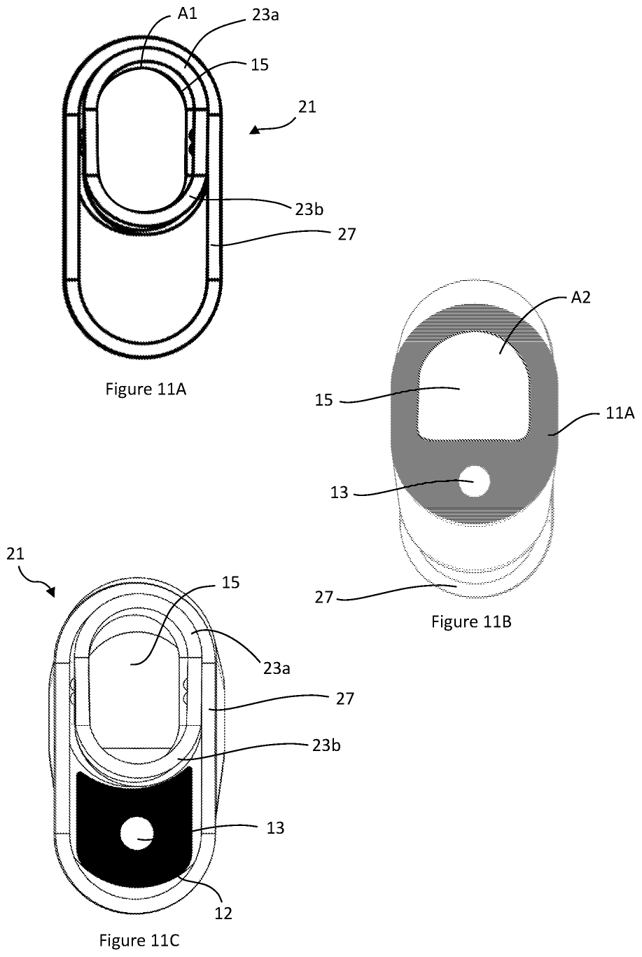

The second portion 11B of the conduit structure 11 comprises a separate fluid supply conduit 12 defining a second portion of the fluid supply lumen 13, and a flexible truss structure 21 defining a first portion of the fluid removal lumen 15. The fluid supply conduit 12 is arranged to be in fluid communication with the fluid supply lumen of the integrally formed portion 11A, preferably with the first and second portions of the fluid supply lumen arranged coaxially. The fluid supply conduit 12 may be an extruded component having fluid impervious walls, for example formed from a material such as silicone. Referring to FIG. 11C, the exterior of the fluid supply conduit 12 may be shaped to complement the truss structure 21.

The flexible truss structure 21 forms the walls of the porous section of the fluid removal conduit 15. The truss 21 is tubular in nature, with a non-circular or circular cross section (in this embodiment the truss defines a lumen 15 with a substantially oval cross section). The truss 21 is configured to, in use, provide support to the surrounding tissue surfaces in all generally radial directions. The truss 21 is flexible in its longitudinal and traverse directions to allow the channel(s) to flex to substantially conform to the contours of the treatment site while having sufficient strength to hold two tissue surfaces apart, at least at the time of implantation, without the truss buckling or the channel collapsing or kinking under movement or application of clinically appropriate levels of negative pressure. The truss 21 is preferably relatively incompressible in the longitudinal direction of the truss 21.

The truss 21 comprises two flexible elongate wall members 23a, 23b, which are wound in a manner to form a framework for, and thereby define, the fluid removal lumen 15 into which fluid from the treatment site can drain or from which fluid can be delivered to the treatment site. The wall members are wound such that they intersect each other periodically at a plurality of cross-over nodes. The wall members 23a, 23b are most commonly helically wound, with the two wall members having opposite (left-hand and right-hand) winds. Alternatively, the truss 21 may comprise helical members wound in the same direction but with different pitches, or a plurality of wall members of an alternative non-helical repetitive shape, such that the wall members periodically intersect each other at cross-over nodes.

The truss 21 further comprises at least two flexible elongate bracing members 25, each bracing member is bonded or linked to the two elongate wall members 23a, 23b at a plurality of the wall member cross-over nodes forming periodic interlocked points along the truss, for example by way of heat bonding. In preferred embodiments, each bracing member 25 extends generally longitudinally along a wall of the outlet lumen 15. These bracing members 25 act to hold the periodic cross-over nodes of the wall members 23a, 23b in spaced apart relation, to reduce or prevent collapse of the lumen walls due to relative movement of these points, thereby preventing or minimising the likelihood of crushing and kinking.

Finally, one or more securing truss members 27 is provided to secure the separate fluid supply conduit 12 alongside the truss 21 defining the removal lumen 15. The securing truss member 27 in the embodiment shown comprises a helical member 27 that is wound about the outside of the fluid removal truss 21 defining the outlet lumen 15 and the separate fluid supply conduit 12. This securing truss member 27 also advantageously spaces the sheath 3 from the impervious wall of the fluid supply conduit 12, thereby creating a fluid path over the wall of the fluid supply conduit 12 to the fluid removal lumen 15. This enables fluid supply to and removal from across the full width of the device 1.

The wall truss members 23a, 23b, and the securing truss member 27, may be tightly wound at respective end portions 24, 28, 29. These tightly wound portions 24, 28, 29 anchor the helical members and facilitate connection between the various device components such as coupling with the integrally formed portion 11A.

To manufacture the truss 21, in a first step, a filament is clamped at one end by a clamp and wound around a first rod-like mandrel in a helical manner at a first pitch length to form part of the first end portion 29. The filament is then further wound around the first mandrel in a helical manner at a second pitch length to form a first wall member 23a with the filament then clamped in place at the opposing end. Two elongate bracing members 25 are then also clamped at their ends by the clamp and laid over the first wall member 23a, along opposing sides of the mandrel, typically top and bottom. A second filament is then clamped by the clamp and wound around the first rod-like mandrel at a first pitch length in the opposite direction to the first wall member 23a to form the second end portion 24. The second filament is then further wound around the mandrel at a second pitch length to form a second wall member 23b. Upon reaching the first part of the first end portion 29, the second filament is then wound at a third pitch length to complete the tight wind of the first end portion 29. The first helical member, bracing members, second helical member and the first rod-like mandrel to form an inner truss.

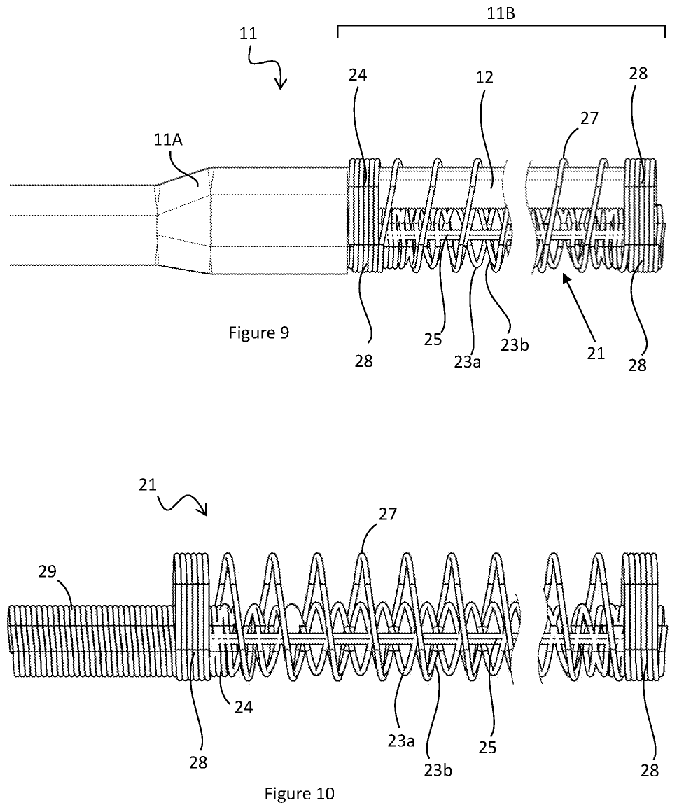

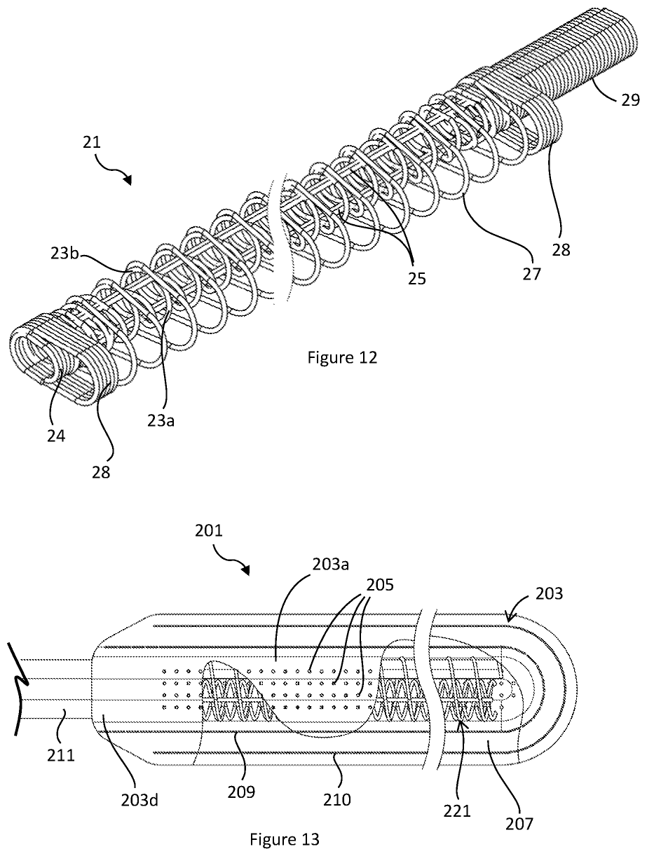

In a next step, a second mandrel is positioned alongside the inner truss and first rod-like mandrel. A filament for forming the securing member 27 is clamped and wound in a helical like manner in a first pitch length to produce a tightly wound first end portion 28. The filament is then further wound in a helical like manner at an increased pitch length to form the securing truss member 27 along a majority of the length of the truss before being tightly wound to form the second end portion 28 at the opposite end to the first end portion 28. The wound filaments are then heated to fuse the bracing members 25 to the first and second wall members 23a, 23b at the points where they overlap. The truss is allowed to cool, thereby setting the shape of the truss members. After cooling the clamp and mandrel are removed leaving the hollow truss as shown in FIGS. 10 and 12. The end 11A of the extruded adjoining removal lumen is pushed over the end portion 29 of the truss structure 21 and the extruded fluid supply lumen 12 is inserted, to form the structure shown in FIG. 9. It will be apparent that the order of the method steps may vary, and that not all steps are necessary.

The truss members 23a, 23b, 25, 27 preferably comprise a non-absorbable polymer filament such as monofilament polypropylene, however, any suitable absorbable or non-absorbable polymer can be used. Preferably the filaments are selected such that the filament can be heated to a melting point without excessive melting occurring that would measurably modify the mechanical properties of the filament.

FIGS. 13 to 15 illustrate a second embodiment device 201, comprising a truss-type conduit structure 221 similar the truss structure 21 described above, but within an alternative form sheath 203. In this device 201, the sheath 203 comprises a single flange 207 that protrudes from a midline of the device, rather than a plurality of tabs 7. The singular flange 207 advantageously allows for better securement when the device 201 is used in undulating sites or sides containing discrete areas that the device cannot be attached to such as bone.

In this embodiment, the open end 203d of the sheath 203 has a constant width and does not narrow. This provides for easier assembly compared to an embodiment that narrows at the inlet.

In devices such as those described above having a linear conduit structure, rather than being sandwiched between two sheets of bioresorbable material, the sheath may comprise a single sheet of bioresorbable material wrapped around the conduit structure and joined along one side the conduit structure. A securement flange may be created along the opposite sides of the conduit by folding the resorbable sheet and sewing along and in from the fold.

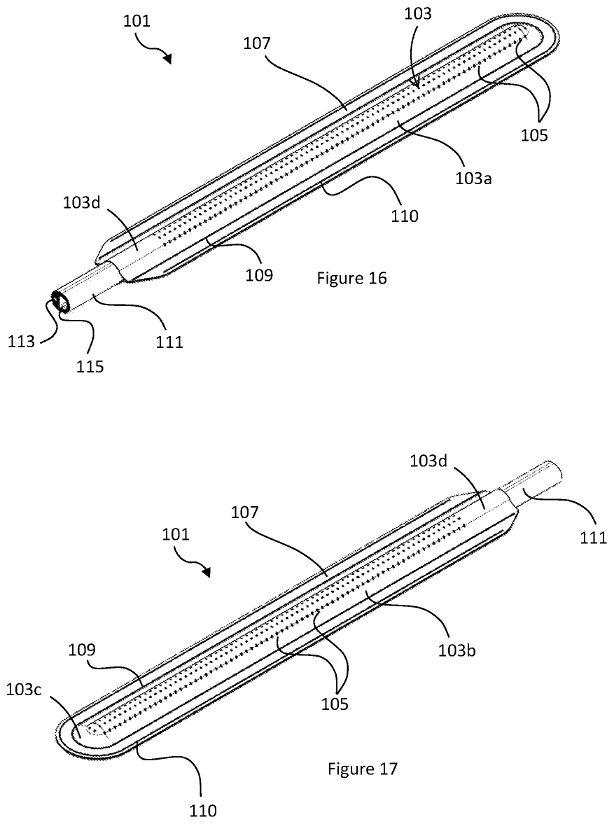

In alternative embodiments, the conduit structure may have alternative forms. FIGS. 16 to 22B illustrate a second embodiment device 101 in which full length of the conduit structure 111 comprises a flexible dual lumen extrusion, in contrast to the truss-comprising structure described previously.

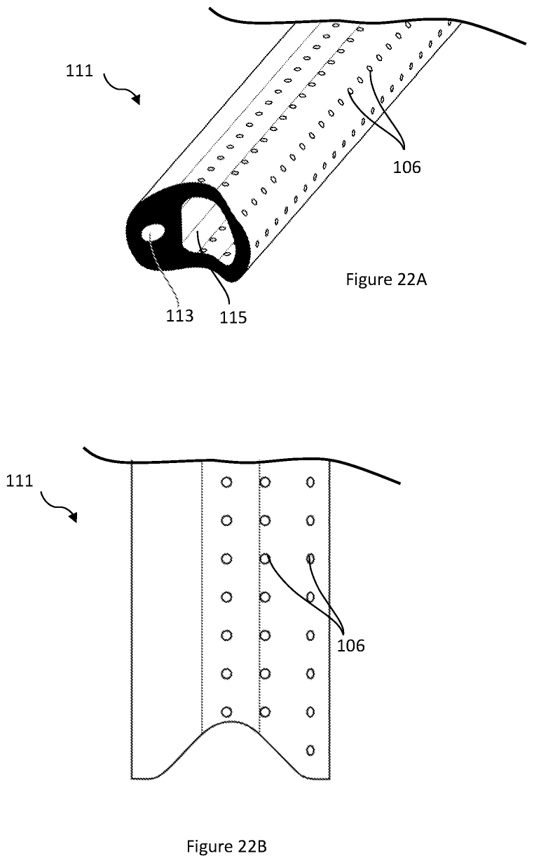

The dual lumen extrusion 111 is typically formed from a material such as silicone, with the fluid supply lumen 115 and fluid removal lumen 113 formed side-by-side. Referring in particular to the section view of FIG. 21, in this embodiment, the fluid supply lumen 113 has a circular cross-section and fluid impermeable walls. The fluid removal lumen 115 is significantly larger than the fluid supply lumen 113 and is D-shaped in cross section. The D-shape provides for an increased removal lumen volume compared with a circular cross-section, given the outer diameter of the conduit structure 111. Apertures 106 are provided in the wall of the fluid removal conduit to allow the egress and ingress of fluid into and from that channel. The apertures 106 each have an area of about 0.5 mm².

The inlet end 103d of the sheath 103 is a constant width and does not narrow towards the device inlet, this facilitates easy removal of the conduit structure 111 when required.

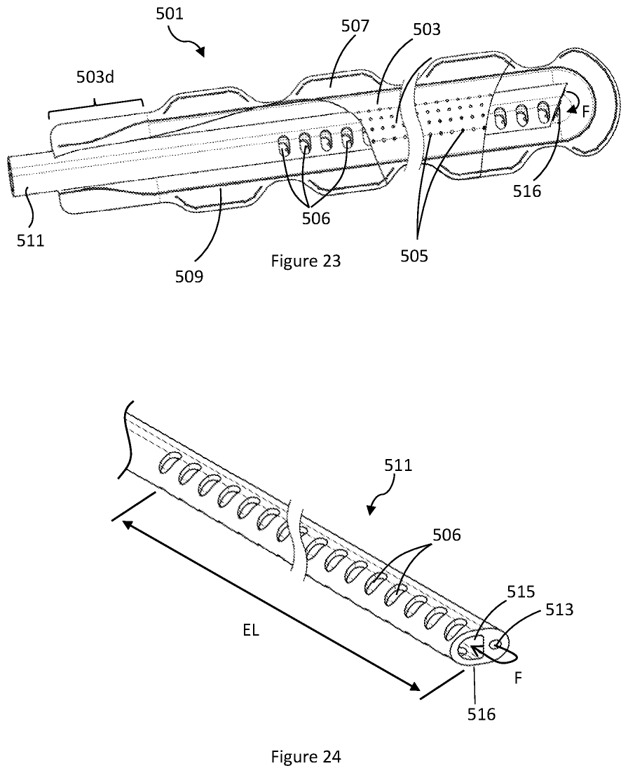

FIGS. 23 to 25(vi) illustrate an alternative form device 501 that is adjustable in length to customise the device to fit various wounds. The device 501 is provided in a first length illustrated in FIG. 23, for shortening as required to fit a smaller treatment site.

This embodiment device 501 comprises a sheath 503 that is substantially the same as the sheath 3 of the first embodiment device 1. The conduit structure 511 comprises a flexible dual lumen extrusion shown in FIG. 24 having a fluid supply lumen 513 with fluid impervious walls, and a larger fluid removal lumen 515. The walls of the fluid removal lumen 515 comprise two oppositely positioned rows of apertures 506 along a length TD of the conduit structure 511. The apertures 506 in this embodiment conduit 511 are larger than those of the previous embodiment 111 and so are more likely to overlap with the smaller apertures 505 of the sheath 503. As such the larger apertures 506 may provide improved exchange of fluid from the treatment site into the conduit.

The apertures 506 are provided along a length EL of the device that is typically shorter than the length of the sheath. This length EL of the device must be contained within a sealed environment (i.e. within the sealed treatment site) to ensure the vacuum is maintained. In addition, for this embodiment, in which the apertures on this conduit are larger than a minimum threshold dimension for preventing blockages (for example, 0.5 mm²), they must also be contained within the sheath 503 of the device 501 to prevent blockages.

The distal end of the conduit structure 511 comprises an angled surface 516, angled to position the outlet of the fluid supply lumen 513 further along the device than the inlet to the fluid removal lumen 515. This angled surface creates a cavity within the sheath 503 between the surface 516 and the end of the sheath 503c, to accommodate fluid flow F from the outlet of the fluid supply lumen 513 to the fluid removal lumen 515.

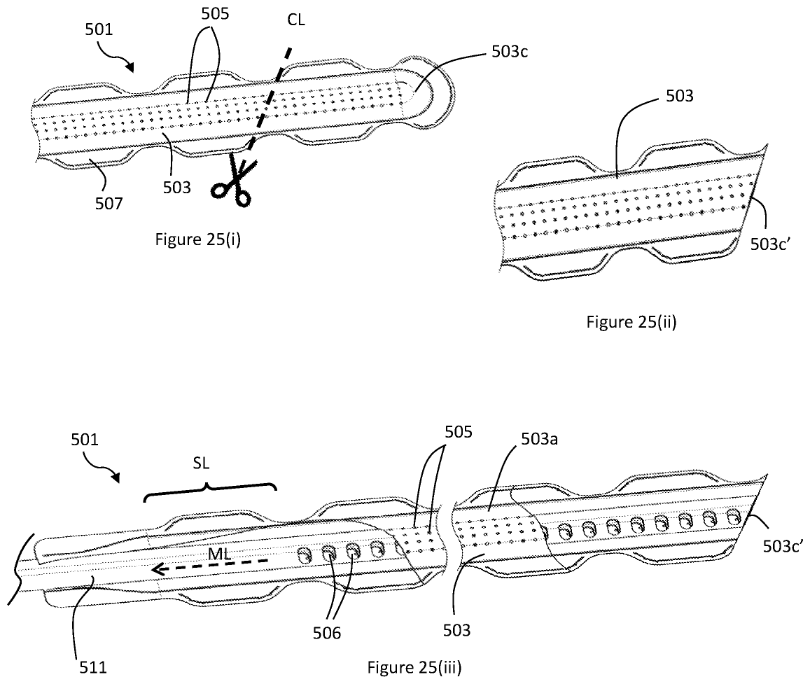

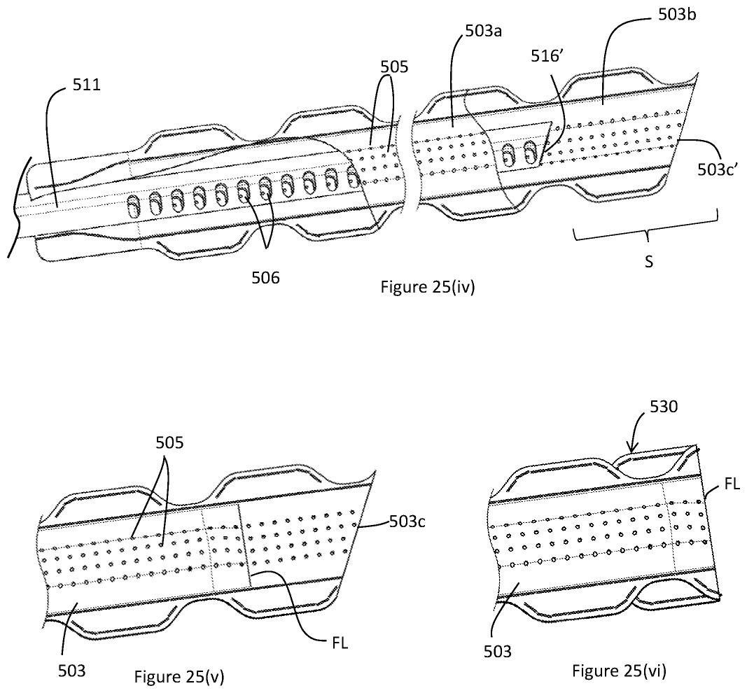

FIGS. 25(i) to 25(vi) illustrate the process of shortening the length of the device 501. In a first step illustrated in FIGS. 25(i) to 25(iii), the device 501 is cut along a cut line CL. The cutline CL is at an angle to the longitudinal direction of the device and should be substantially parallel with the angled end 516 surface of the conduit structure 511. Cutting the device 501 at an angle in this manner ensures that the shortened device will retain the cavity within the sheath 503 between the surface 516 and the end of the sheath to accommodate fluid flow F. The position of the cutline CL should be selected to be slightly longer than the desired length of the device to accommodate the sealing of the sheath end as will be described below.

In a second step, the conduit structure 511 is pulled in the direction of the inlet, as illustrated by the like ML of FIG. 25(iii), relative to the sheath. This creates a spacing S between the cut, angled end 516′ of the conduit structure 511 and the cut end 503c′ of the sheath 503 as illustrated in FIG. 25(iv). To accommodate this anticipated movement of the conduit structure 511 within the sheath to resize the device, the conduit structure may have a sealed length SL free from apertures 506. This length SL ensures the large apertures 506 are not pulled beyond the sheath or to a position that may compromise the seal of the device or result in blockages when the device is shortened.

To mitigate this, and to provide a device with a longer effective length EL, small apertures having a dimension smaller than a blocking threshold may optionally be provided along the length SL. A longer effective length EL is advantageous because it increases the effective treatment area of the device, which is determined by distance to the nearest aperture 506. As one example, in the embodiment of FIG. 23, the device may only provide treatment to within about 25 mm to the nearest aperture.

Referring to FIGS. 25(v) and 25(vi), in a final step, the excess material 530 at the end of the sheath 503 is flattened and folded over along a perpendicular fold line FL to close the end of the sheath. The folded portion 530 is secured to tissue at the treatment site to prevent any unwanted tissue ingress into through the cut end of the device.

In one embodiment, the device illustrated in FIGS. 25(i) to 25(vi) may be provided as separate components for assembly by a clinician before use. In such an embodiment, the sheath 503 may be in a generally tubular form having two open ends, with the proximal section 503d substantially as described herein in relation to the various embodiments, with the opposing distal end 503c open, in a form similar to that shown in FIG. 25 (ii). The open end at 503c may have any suitable shape, such as a square edge or an angled edge. The end edges of the top and bottom sheaths may align (as shown in FIG. 25(ii), or one sheath of the device may extend beyond the other sheath to facilitate assembly with the conduit structure. This alternative embodiment device may be assembled following the steps shown in FIGS. 25(iii) to 25(v) within the clinical setting prior to implantation, with the distal end of device 503c folded over and secured place (as shown in FIG. 25(vi)) during implantation.

FIGS. 22A and 24 in particular exemplify two alternative non-resorbable dual-conduit type structures for use in the devices of FIG. 23. However, alternative designs are envisaged. In the embodiment of FIGS. 23 to 25(vi) the fluid removal lumen 515 has a D-shaped cross-sectional with an area of about 18 mm², and the fluid supply lumen has a circular cross section with a diameter of about 1.4 mm. However, other sizes and shapes for these lumens are envisaged. For example, the inlet lumen may have a diameter from about 1 mm to about 2 mm.

In embodiments where the conduit structure 111 of FIGS. 22A and 22B is used, the small 0.5 mm apertures 106 enable the apertures to cover a longer length of the device. When adjusting the length of the device, the conduit structure 111 may be pulled so that some of the apertures are in the sealing region 503d of the sheath, or even outside of the sheath, but the small size of the apertures prevents the blocking of the fluid removal lumen.

In a further embodiment, small sized apertures 106 may only be provided in a localised zone near the sealing end 503d of the sheath (but internally in the sheath). The remaining length of the fluid removal conduit may include larger apertures to provide a higher degree of fluid exchange between the conduit and the treatment area. This mean that if a user inadvertently removes too much of the tube from the sleeve during the steps to shorten the device such that some of the apertures are outside of the sheath, the small size of the apertures prevents this resulting in blocking of the fluid removal lumen.

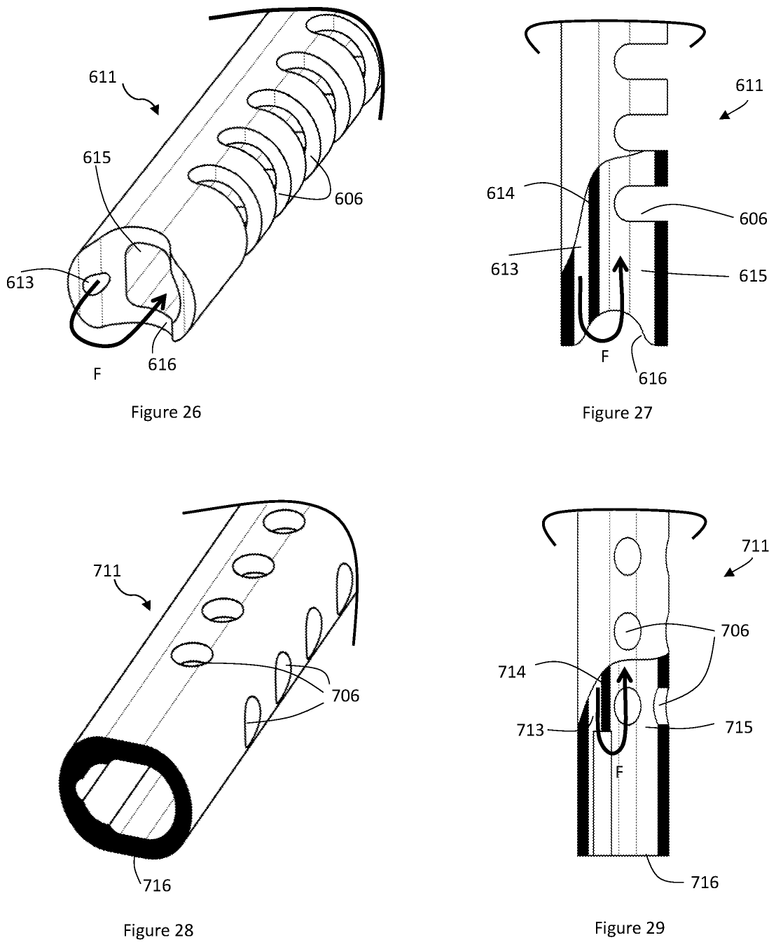

As examples, FIGS. 26 to 35 illustrate some further embodiment conduit structures. FIGS. 26 and 27 illustrate one embodiment conduit structure 611 having a fluid supply lumen 613 and a fluid removal lumen 615, separated by an internal call 614. In this embodiment, the fluid removal lumen 615 comprises a row of slot-like apertures 606. The apertures 606 follow the curvature of the lumen wall and their larger size improves the passage of fluid from the treatment site into the fluid removal lumen 616. The end face 606 of the conduit 611 has a concave surface 616 that extends at least partly across the respective ends of the fluid supply and fluid removal lumens 613, 615. This concave surface creates a cavity within the sheath between the concave surface 616 and the end of the sheath, to accommodate fluid flow F from the outlet of the fluid supply lumen 613 to the fluid removal lumen 615, ensuring that neither the outlet of the fluid supply lumen 613 of the inlet of the fluid removal lumen 615 is blocked by the sheath.

FIGS. 28 and 29 illustrate another embodiment conduit structure 711. In this embodiment, the internal wall 714 separating the fluid supply lumen 713 and the fluid removal lumen 715 terminates before the end of the conduit 716. This setting back of the dividing wall 714 provides a passage for fluid to flow F between the two lumens within the conduit 711, at a point spaced from the conduit end wall 716. This ensures that the passage between the lumens 713, 715 is maintained if the sleeve is sucked against or into the exposed end of the conduit 716 during use.

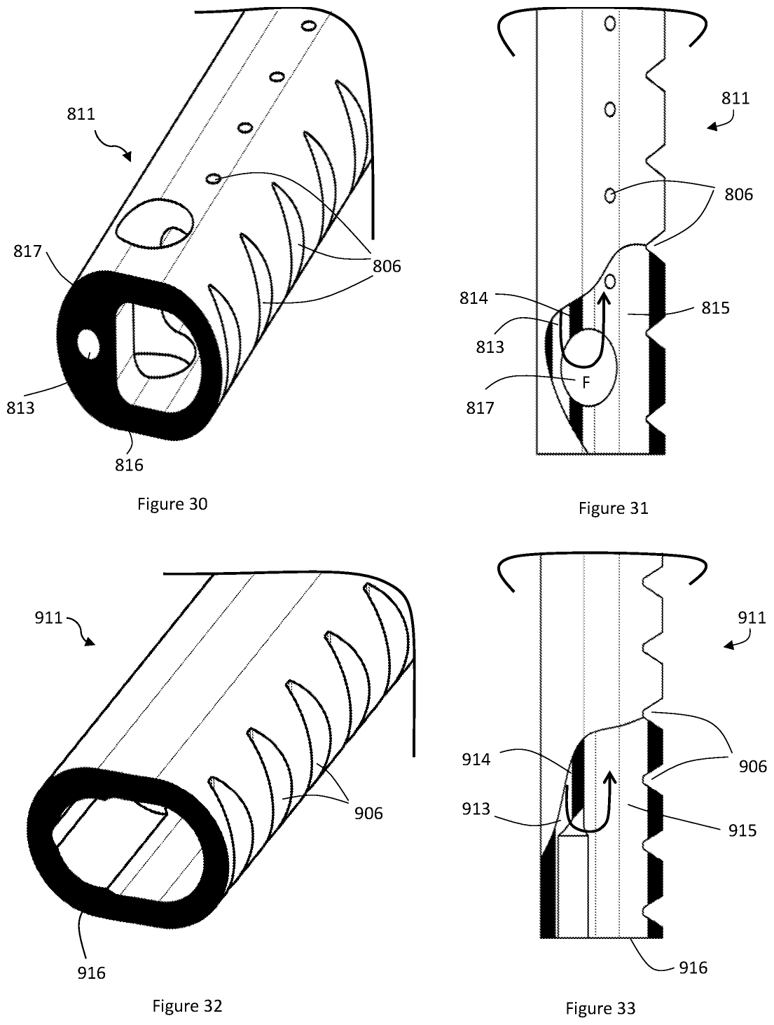

FIGS. 30 and 31 illustrate a further embodiment conduit structure 811. In this embodiment, apertures 806 along the fluid removal conduit are provided by both rows of apertures and a row of slits. The apertures 806 are elongate, slit-like apertures, V-shaped in profiles. A through hole 817 is provided through the conduit, perpendicular to the longitudinal direction of the conduit 811, at a point spaced from the conduit end 816. This through hole extends through the internal wall 814 separating the fluid supply lumen 813 creating a passage for fluid to flow F between the two lumens within the conduit 811, at a point spaced from the conduit end wall 816. This ensures that the passage between the lumens 813, 815 is maintained if the sleeve is sucked against or into the exposed end of the conduit 816 during use.

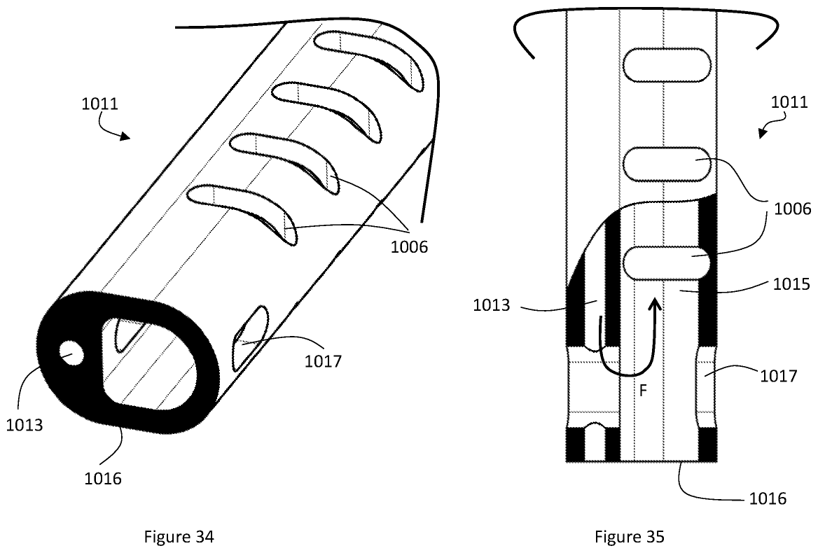

FIGS. 32 and 33 and FIGS. 34 and 35 illustrate two further embodiment conduits 911, 1011 illustrating variations of the previously described embodiment. The conduit of FIGS. 32 and 33 comprises a shortened internal wall 914, and a row of slit-like apertures along the fluid removal conduit 915. The conduit of FIGS. 34 and 35 comprises a transverse through hole 1017, and dual rows of slit-like apertures 1006. It will be appreciated that features may be selected from different ones of the embodiments described above and combined as desired to create further embodiments.

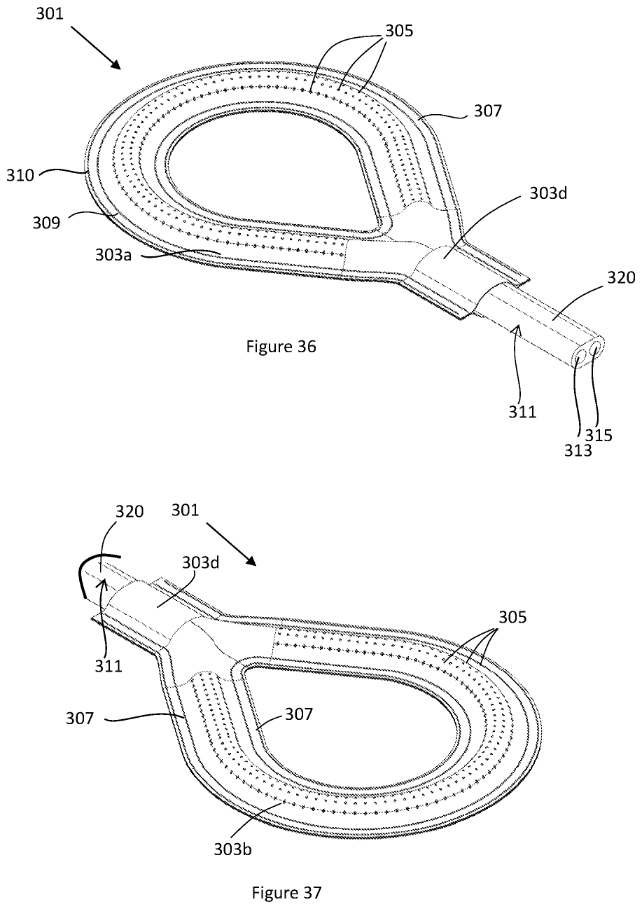

FIGS. 36 to 40 illustrate an alternative form device 301 that may be particularly suitable for large wounds. In some applications this embodiment device 301 may be placed along a plane of tissue, for example secured to a fascia of repaired muscle, or across a plane of repaired tissue, for example, incorporated into a repair across a layer of tissue such as the closure of the sub-cutaneous layer of tissue. In this embodiment, the fluid supply lumen 313 and the fluid removal lumen 315 are coaxial and form a loop.

The conduit structure 311 in this embodiment 301 comprises a coupling inlet/outlet component 320 of the device 301, which defines a portion of the fluid supply lumen 313 and an outlet portion of the fluid removal lumen 315. The component 320 is Y-shaped, with the lumens 313, 315 being generally parallel and side-by-side for a length through the component, then diverging at the end of the component 320 distal to the inlet/outlet ports. The lumens 313, 315 preferably have the same cross-sectional shape and size. In this example the lumens 313, 315 are circular in cross-section, but other shapes are envisaged.

The diverging ends of the coupling inlet/outlet component 320 are configured to fluidly couple to a conduit structure 321 that forms the framework for the remaining length of the fluid removal lumen. Therefore, in this embodiment, the fluid supply lumen 313 extends only through the coupling component 320 (and optionally any upstream coupled component or a short length of the conduit structure 321) and so is much shorter in length than the fluid removal lumen which extends around a majority or all of the loop as well as the portion 315 through the coupling component 320.

The walls of both the fluid supply and fluid removal lumen portions 313, 315 in the coupling component 320 are substantially fluid impervious such that 100 percent of fluid supplied to the device 321 is supplied to the looped portion 321 of the conduit structure 311. The coupling component 320 may be a moulded component, for example, moulded from silicone.

If the spacing between the fluid supply point (ie the terminal end of lumen 313 in this example) and the terminal end of the lumen 315 is too close, supplied fluid may bypass the looped portion 321 and thereby the majority of the treatment site and instead be drawn straight out of the device 301. Therefore, these ends should be sufficiently spaced apart.

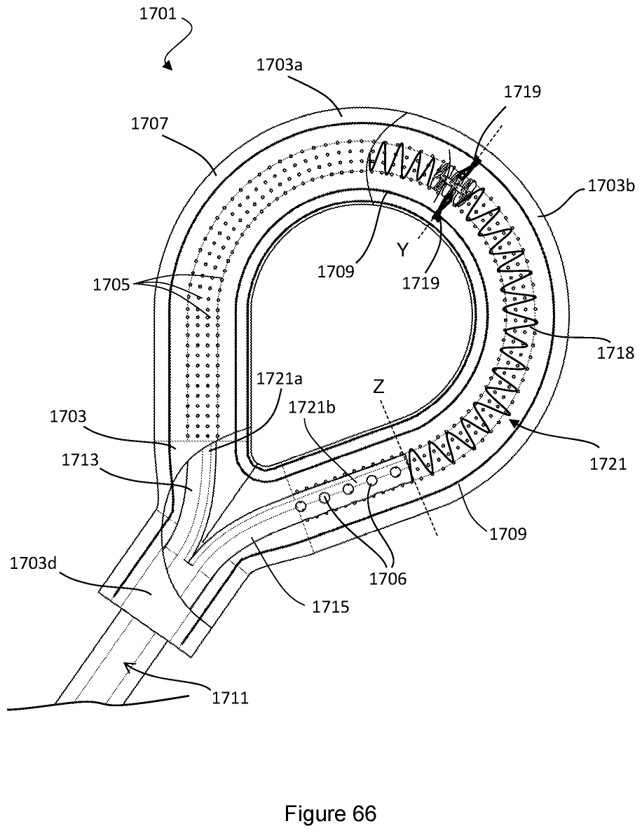

Any conduit structure defining an elongate channel may be suitable for use in the looped portion 321 of the conduit structure 311, for example, a truss-based or hollow extrusion type conduit structure. The structure 321 may comprise bioresorbable material that doesn't require removal from the wound site, or it may comprise a non-resorbable material such that the structure 321 will be removed once treatment is completed. In some embodiments such as the one illustrated in FIG. 66 and described in more detail below, the conduit structure may have different forms along the length of the channel. For example, one or more portions of the conduit structure may be bioresorbable and one or more other portions may require removal. One or more portions of the conduit structure may have a truss-based structure, and one or more other portions may have a hollow extrusion-type structure, for example.

The looped structure advantageously enables the application of vacuum pressure to the centre portion of the device which applies vacuum pressure directly to the central area of the treatment site which is positioned the furthest away from the edges of the defect site (which has the highest likelihood of moving or remaining detached).

In the embodiment shown 301, the conduit structure 321 comprises two lengths 321a, 321b, a first length 321a joined to the inlet lumen 313 of the coupling component 320, and a second length 321b joined to the outlet lumen 315 coupling component 320. The distal ends of the two lengths are substantially butted together at a join 315. They may be held in this position by the surrounding sheath, or utilising a connector component internally positioned in the conduit lumen or externally positioned around the conduit structure 321. In alternative embodiments, the conduit structure 321 may comprise a single length, with a first end joined to the inlet lumen 313 of the coupling component 320, and a second end joined to the outlet lumen 315 coupling component 320.

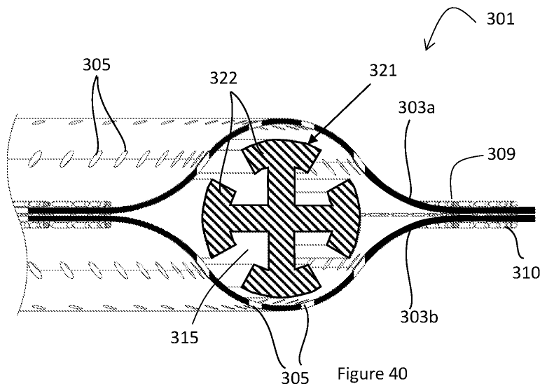

FIG. 40 illustrates one form of conduit structure 321 for use in the embodiment of FIGS. 36 to 39. The structure 321 is formed from a non-resorbable material such as silicone and comprises an extrusion having an X-shaped cross section The X-shape defines four flow paths along the fluid removal lumen 315. Curved flanges 322 on the ends of each cross member act to support the sheath 303 sufficiently to prevent the sheath being drawn into the fluid removal conduit upon the application of negative pressure. The flanges 322 define four elongate slit-like openings into the four respective passages to allow for the passage of supplied fluid out of the lumen and for the passage of wound fluids into the lumen 315.

In the device 301 of FIGS. 36-40, the sheath 303 encompassing the structure 321 comprises a top sheet 303a and a bottom sheet 303b, with a single flange 307 formed around the outer perimeter of the device and a second internal flange with an opening in the centre of the loop. Apertures 305 are provided in both the top and bottom sheath sheets 303.

The sheath forms an inlet portion 303d free of apertures that wraps over the Y-shaped coupling 320.

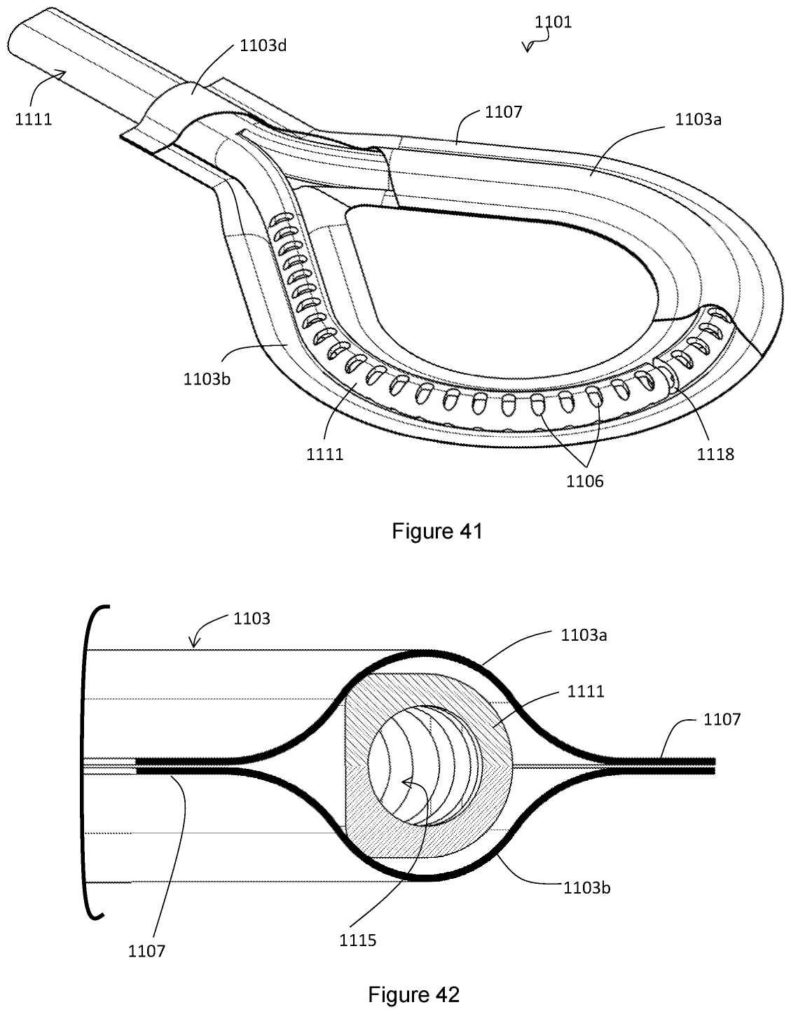

FIGS. 41 to 45 and 46 to 51 illustrate two alternative loop-type embodiments 1101, 1201. In these embodiments, the separate Y-shaped connector is omitted, and instead the conduit structures 1111, 1211 are manufactured as an integral component with a dual lumen portion 1111a, 1211a splitting at a junction into two separate single-lumen limbs 1111b, 1211b, 1111c, 1211c. Each limb 1111b, 1211b, 1111c, 1211c comprises a plurality of apertures 1106, 1206 for the exchange of fluids through the wall of the structure 1111, 1211.

FIGS. 45 and 51 illustrate the respective conduit structures 1111, 1211 before assembly of the device 1101, 1201. To assemble the device, the ends of the conduit structure limbs 1111b, 1211b, 1111c, 1211c are butted together at a join 1118, 1218 to be coaxial, forming a single continuous lumen through the device 1101, 1201. In this embodiment, the sheath holds the limbs in position when the two sheets are stitched together around the conduit structure. Alternatively, an additional sleeve of bioresorbable material may be tightly wrapped around the conduit limbs where they are butted together 1118, 1218 to firmly maintain the conduits in position. For those skilled in the art other means for maintaining these conduit limbs in position is envisaged.

The aperture arrangements 1106, 1206 on the conduit structures 1111, 1211 of these two embodiments 1101, 1201 are examples only, and many different aperture shapes and layouts will be possible. For each limb of the conduit structure, the wall of a first length of the conduit adjacent the Y-junction is free from apertures 1106, 1206 such that no fluid transfer into or from the lumen is possible over that length. These aperture-free portions of the limbs 1111b, 1211b, 1111c, 1211c are important to create a spacing S between the first point at which supplied fluid can migrate through the sheath 1103, 1203 to the wound site, and the first point at which fluid can be drawn from the wound site. If this spacing S is too close, supplied fluid may bypass the looped portion and thereby the majority of the treatment site and instead be drawn straight out of the device 1101, 1201.

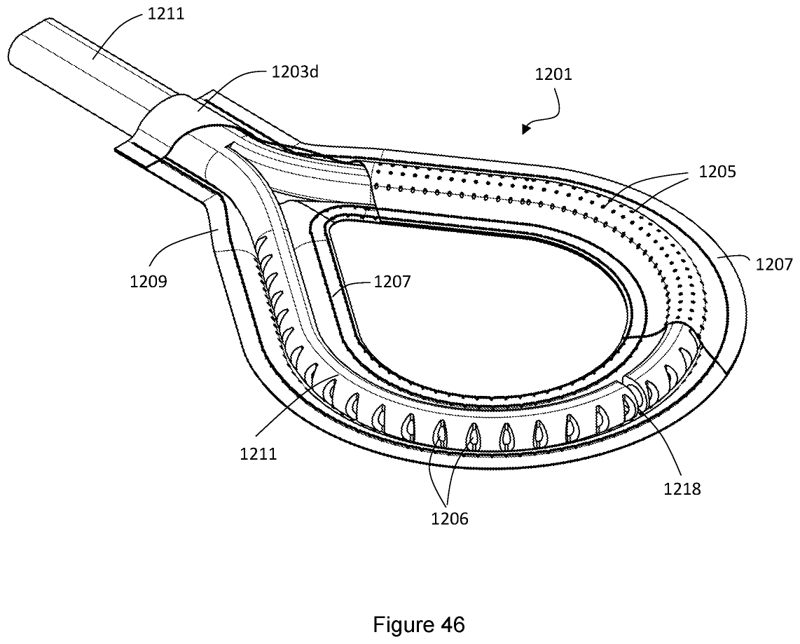

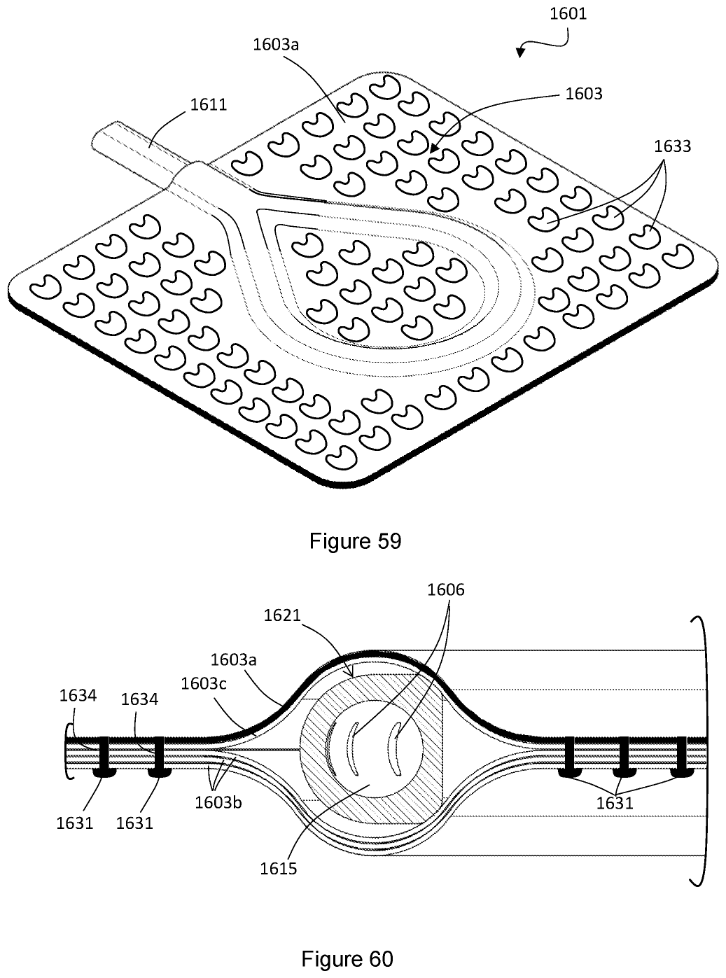

As for the above embodiments, the sheath 1103, 1203 may comprise apertures, or may be free from apertures and rely on the porosity of the sheath for fluid transfer. In the embodiment 1101 shown in FIG. 41, the sheath is free from apertures and relies on the porosity of the sheath to facilitate fluid transfer across the sheath 1103. FIG. 59B illustrates flow AF across a processed layer of ECM material. The sheath 1203, in the embodiment of FIGS. 46 to 48 comprises multiple rows of apertures on both the top and bottom sheath sheets 1203a, 1203b.

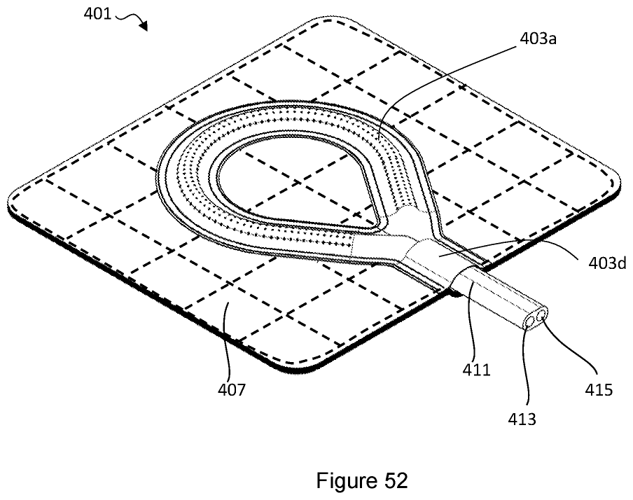

FIGS. 52-54 show a further embodiment 401, where the fluid drainage and supply device is incorporated with a multi-layer reinforced surgical mesh. The surgical mesh may be resorbable or non-resorbable. The surgical mesh forms the lower layer of the sheath 403b. A top sheet of material forms the top layer of the sheath 403a. The number of sheets 403a, 403b and the distribution of sheets on top of and below the conduit structure 421 may vary in different embodiments. Apertures 405 are provided on the top layer 403a of the sheath. This top layer 403a may cover the entire surgical mesh or may be shaped to only cover the conduit structure 411. The multilayer surgical mesh is reinforced using stitching 410 in a pattern that accommodates the conduit structure 411 and ensures the conduit structure 411 can be removed when treatment is concluded.

This embodiment 401 may have particular application for abdominal wall repair. If used, for example, in a complex hernia repair the sheath apertures 405 would typically face towards the skin and away from the abdominal cavity. This advantageously ensures the vacuum is applied to the separated tissues that lay above the device, ensuring effective fluid removal and the removal of dead space. The underside of the device 403b is free from apertures, which can aid in the healing of abdominal wall by preventing the application of vacuum pressure resulting in the underlying tissue adhering to the surgical mesh. While the apertures 405 the top sheath layer 403a act to improve the apposition of the separated subcutaneous tissues to the device 401 to diminish the clinical dead space that remains following the completion of the surgery.

In an alternative embodiment multiple conduits and/or upper sheath layers may be fixed to a single mesh.

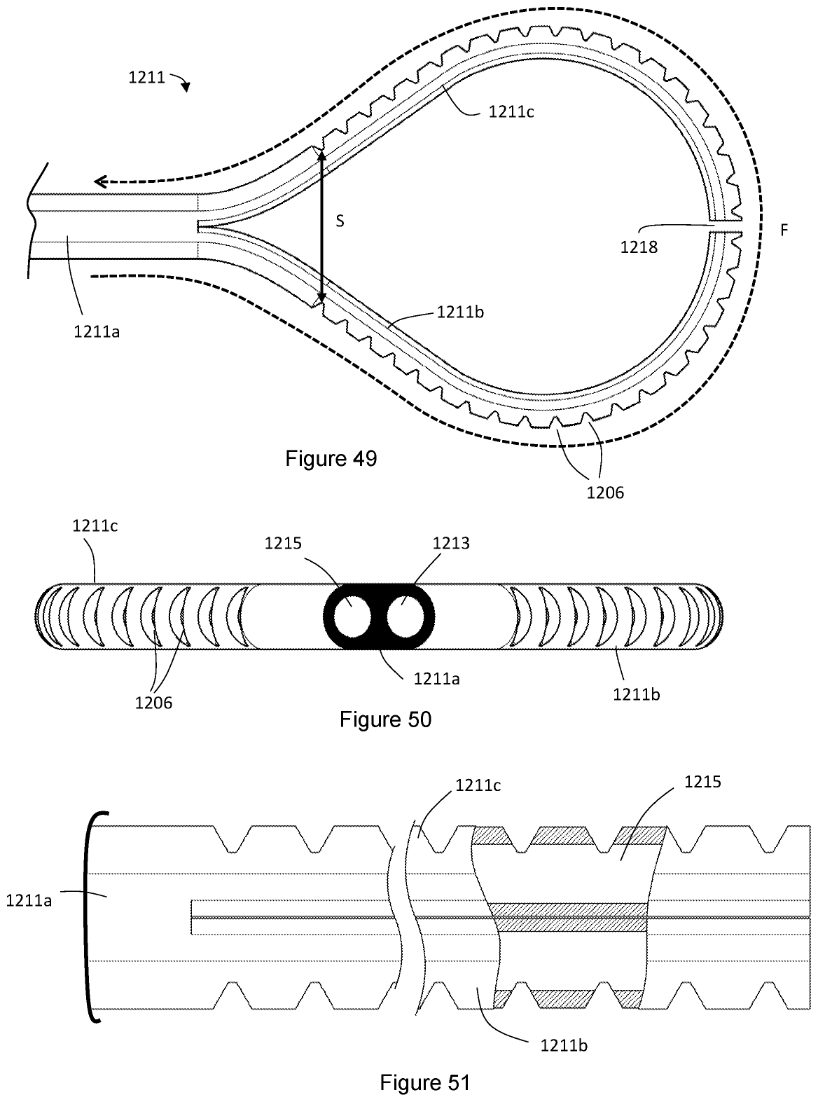

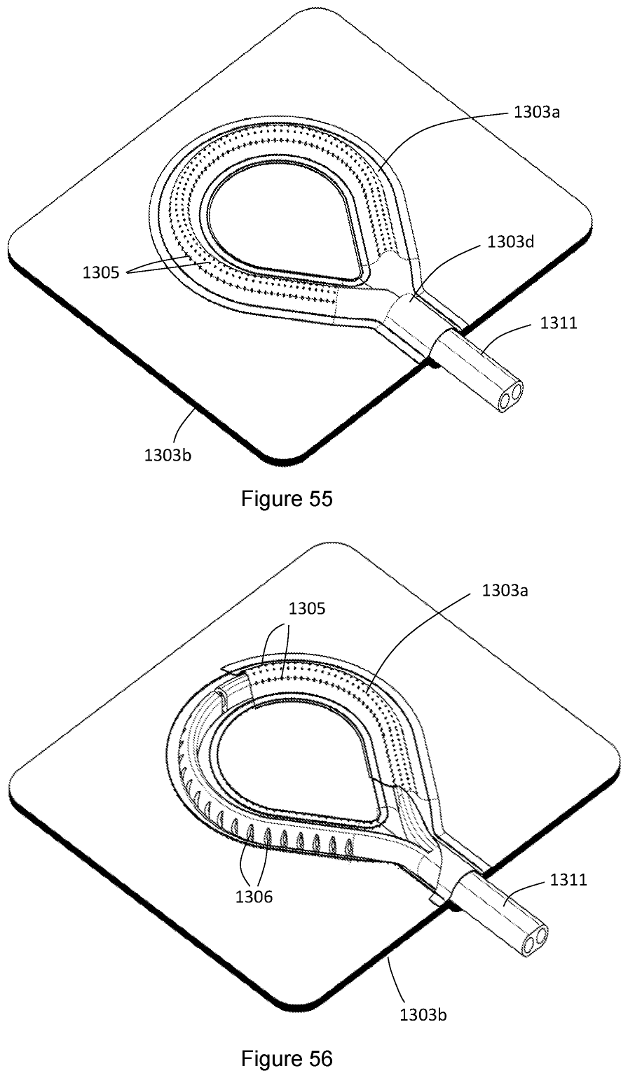

FIGS. 55 to 57 illustrate a similar embodiment device but having an alternative conduit structure 1311 that is substantially as described with respect to FIGS. 49-51. In this embodiment the top of the sheath 1303a has a series of apertures, where the underside of the sheath 403b is free of holes. The lower sheath 1303b of the device 1301 could be formed from one or more layers of polymeric material, for example, ECM, polymer, foam etc, for use as an implant or as a cover to achieve a vacuum seal over a wound.

FIGS. 59 and 60 illustrate a further embodiment device 1601 in which the sheath 1603 is formed by multi-sheet structure comprising a plurality of sheets mechanically interlocked together. This multi-layer structure of the bioresorbable layer may be produced according to the method described PCT application PCT/NZ2015/050215, which is incorporated herein by reference.

In the embodiment shown, the top layer of the sheath 1603 is formed from a first, lugged sheet 1603a having a plurality of lugs 1631 formed by cutting a U-shaped or C-shaped slit in the first sheet to create a tab-like ‘lug’, and optionally one or more underlying sheets 1603c. The lower layer of the sheath 1603 comprises a plurality of sheets 1603b, each having a plurality of aligned perforations 1634. Each lug 1631 is pushed through the respective underlying perforations to interlock the sheets together to create a lugged laminate structure. The resulting structure contains recesses 1633 in the top lug sheet where each lug 1631 was cut from the sheet. Each lug 1631, remains attached to the lug sheet, via a connection bridge, thereby interlocking the sheets to hold them together.

In the exemplary embodiment there is one lugged sheet and four perforated sheets, with one perforated sheet being positioned over the conduit structure 1621. However, alternatively the sheath 1603 may comprise more or fewer perforated sheets, and optionally may include more than one lug sheet 1603a. The number of sheets 1603a, 1603b, 1603c and the distribution of sheets on top of and below the conduit structure 1621 may vary in different embodiments. The lugs 1631 may or may not be pushed through all of the underlying or overlying sheets and, for embodiments with more than one lug sheet, may or may not be pushed through the other lug sheet.

The perforations 1634 for the lugs provide a plurality of micro-channels through the sheet. These channels advantageously assist with fluid flow from the wound through the bioresorbable layer and assist with pressure application to the wound due to the channels provided by the perforations for the lugs.

The sheets of the multi-layer sheath preferably comprise extracellular matrix (ECM) or a polymeric material. ECM-derived matrices for use in embodiments of the present invention are collagen-based biodegradable matrices comprising highly conserved collagens, glycoproteins, proteoglycans and glycosaminoglycans in their natural configuration and natural concentration. One extracellular collagenous matrix for use in this invention is ECM of a warm-blooded vertebrate. ECM can be obtained from various sources, for example, gastrointestinal tissue harvested from animals raised for meat production, including pigs, cattle and sheep or other warm blooded vertebrates. Vertebrate ECM is a plentiful by-product of commercial meat production operations and is thus a low cost tissue graft material. One exemplary method of preparing ECM is described in U.S. Pat. No. 8,415,159.

In some embodiments of the invention, resorbable polymeric material may be included in the bioresorbable layer as either lug sheets, pierced sheets, and/or in another three-dimensional form. For example, meshes comprising synthetic materials such as polyglycolic acid, polylactic acid and poliglecaprone-25 are will provide additional strength in the short-term, but will resorb in the long term. Alternatively, the polymeric material may be a natural material, or derived from a natural material, such as proteins (e.g. collagen), polysaccharides (e.g. alginate), glycoproteins or other materials.

As illustrated by the exemplary embodiment 1401 in FIG. 58, the conduit structure 1411 incorporated into a multi-layer product or structure may follow any desired path. For devices that cover larger areas, snaking of the conduit structure, as illustrated in FIG. 58, may be desirable to increase the length of the fluid removal lumen and thereby to increase the area across which fluid and/or negative pressure is delivered.

Greater conduit coverage across the surface of the surgical mesh will increase the total force supplied to the treatment site, which improves the likelihood of achieving complete dead space closure and fluid removal and is likely to improve the clinical outcomes of the treatment.

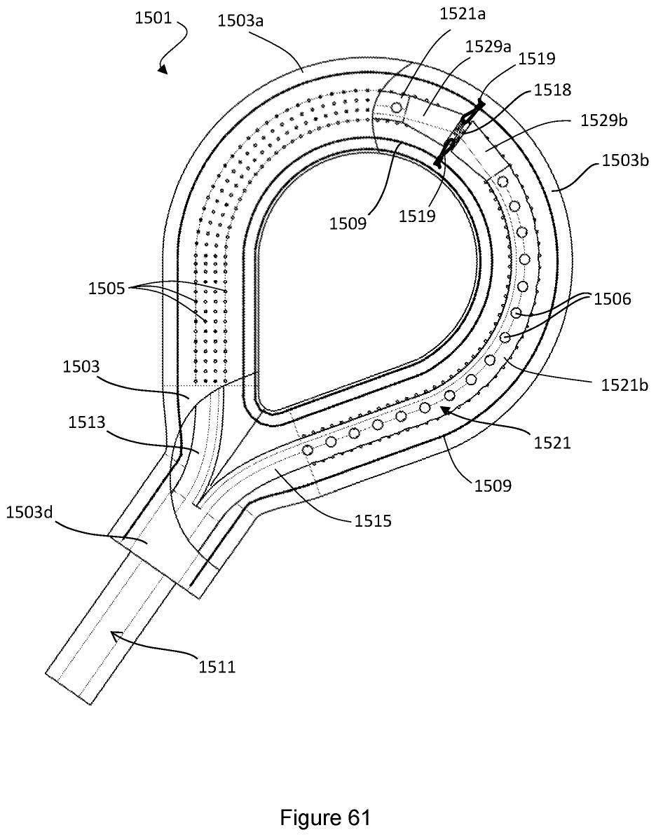

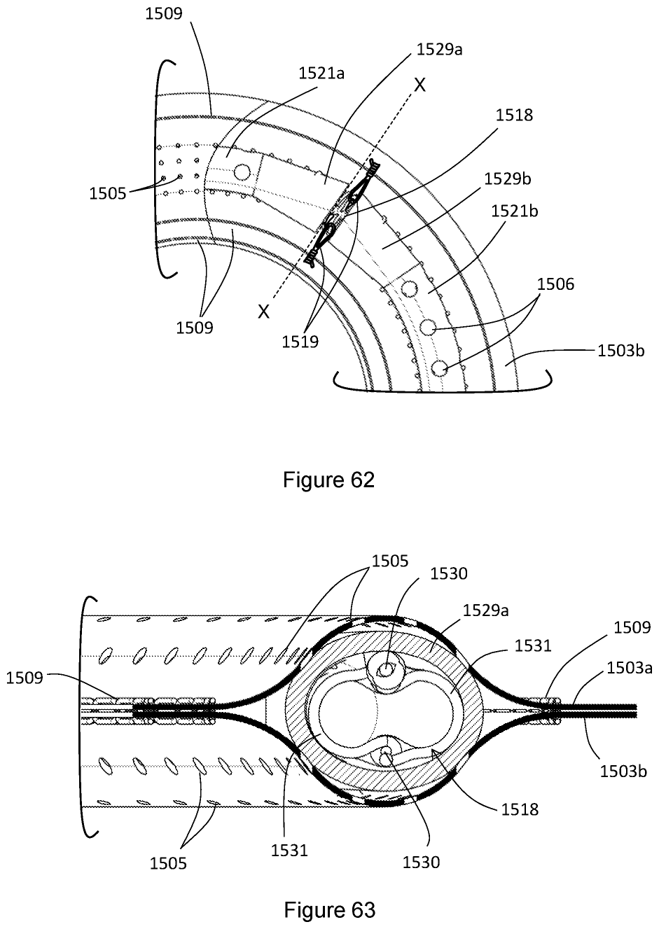

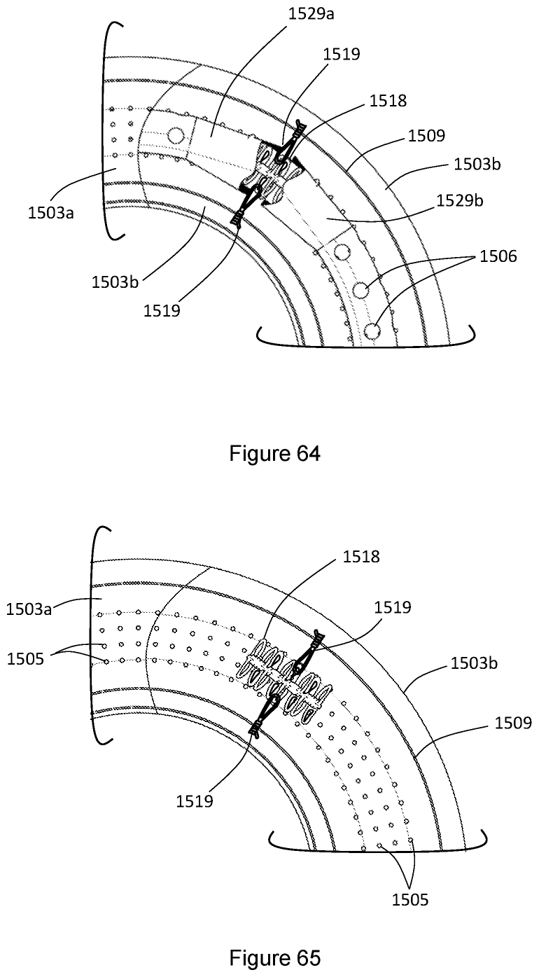

FIGS. 61-65 illustrate a further alternative form device 1501 having a looped structure. The conduit structure of this embodiment comprises two limbs 1521a, 1521b that join at their ends to create a continuous lumen along the conduit structure 1521. The respective ends of the two limbs 1521a, 1521b may be butted together so they contact or there may be a small spacing between the ends as shown in the exemplary embodiment 1501. The respective ends of the two limbs 1521a, 1521b are joined using an internal connector 1518. A portion of the connector 1518 fits snuggly in the lumen of an end portion 1529a of the first limb 1521a and a second portion of the connector 1518 fits snuggly in the lumen of an end portion 1529b of the second limb 1521b.

In the embodiment shown, the connector 1518 is a bioresorbable component in the form of a bioresorbable resilient truss structure. The applicant's earlier applications PCT/NZ2018/050134 and PCT/NZ2021/050206, herein incorporated by reference, describe some exemplary truss structures. The truss structure may comprise one or more elongate truss members wound to form a flexible tube thereby defining an internal channel or lumen. The truss member(s) may comprise one or more substantially helical members, for example, a first substantially helical truss member with a first pitch length, and a second substantially helical truss member wound in the same or the opposite direction with a second pitch length that may be the same or different to the first pitch length.

The truss structure may comprise one or more elongate bracing members joined to the one or more bracing members at discrete points to strengthen the truss structure. The bracing member(s) may be heat welded or adhered to the truss member(s) or may be mechanically linked with the bracing member(s) such as by twisting or looping the members together as described in PCT/NZ2021/050206.

In the exemplary embodiment connector 1518, the truss structure comprises two truss members 1531a, 1531b and upper and lower bracing members 1530. The truss members are twisted around the bracing members to secure the structure. Each of the two truss members alternates from being on a left side of connector to being on the right side of the connector. The twisted portions of the truss members cover substantially the whole length of each bracing member.

The shape of the connector 1518 may be substantially cylindrical or oval or elliptical or it may have another shape to be compatible with the conduit structure 1521 of the device. In the embodiment shown, the connector 1518 has a substantially oval cross-section and defines a channel therethrough with a correspondingly oval cross-section.

To join the two limbs 1521a, 1521b, one end of the truss-based connector 1518 is inserted into the end portion 1529a of the first limb 1521a the opposite end of the connector 1518 is inserted into the end portion 1529b of the second limb 1521b. The fit between the respective conduit limb and the connector 1518 is a snug, push fit, to resist inadvertent decoupling of the components. In the embodiment shown, the conduit structure 1521 is substantially cylindrical, but the end portions 1529a, 1529b deform to match the oval cross section of the connector 1518 where they engage the connector 1518. Therefore, the cross-section of the fluid removal lumen in the embodiment of FIGS. 61-65 transitions from being circular along the majority of the first limb 1521a of the conduit structure to being oval along the connector 1518, to being circular along the majority of the second limb 1521b.

In the exemplary embodiment, the end portions 1529a, 1529b of the conduit limbs are free from conduit apertures 1506. Conduit apertures in the end portions 1529a, 1529b may compromise the strength of the connection or may introduce a risk of wall of the conduit tearing. However, in alternative embodiments, the end portions 1529a, 1529b of the conduit limbs may include apertures, the configuration of which may match the remaining portion of the respective conduit limb, or the configuration of the apertures may be varied in the end portions.

In one embodiment, the connector is secured in the two conduit limbs and not otherwise connected to the device 1501. At the conclusion of treatment, the non-resorbable limbs of the device are tugged to draw the limbs out of the wound. This causes at least one of the conduit limbs to detach from the connector so they can be drawn out. The connector may remain retained in the other conduit limb and be pulled from the wound with that respective conduit limb. If both conduit limbs detach from the connector during the removal process, the connector may remain in the wound to be resorbed.

Alternatively, the connector may be secured to the body of the bioresorbable device 1501 to prevent it being pulled from the wound at the conclusion of treatment. Referring to FIG. 60, the connector 1518 may be tied or stitched to the body of the device. In this embodiment, retaining loops 1519 are secured to the truss of the connector 1518 and to one or more rows of stitching 1509. The stitching the retaining loop is connected to may be the row(s) of stitching 1509 securing the top and bottom layers 1503a, 1503b of the sheath, or it may be a separate row of stitching.

The retaining loop 1519 may comprise a natural or synthetic absorbable or non-absorbable suture material, such as collagen sutures, polypropylene, polyglycolic acid, polydioxanone, poliglecaprone-25, or polyester etc.

FIG. 66 illustrates a further embodiment device 1701 in which the ends of the two limbs 1721a, 1721b, are spaced apart and connected by an elongate truss-based connector 1718. The elongate connector 1715 forms a length of the conduit structure 1721, defining a respective length of the fluid removal lumen having a porous wall for the transfer of fluid into said lumen. The connector 1721 is illustrated schematically in FIG. 66 but may comprise any suitable truss structure such as those described and referenced above.

In this embodiment 1701, the elongate connector 1718 may be configured to decouple from both conduit limbs 1721a, 1721b upon initiation of removal of the two limbs from the treatment site at the completion of treatment such that the connector 1718 remains in the wound. It may be optionally secured to the sheath of the device, for example with ties 1719, to inhibit removal of the connector 1718.

This embodiment 1601 may facilitate easier removal of the conduit limbs 1721a, 1721b as it necessitates removal of a shorter length of conduit from the wound. The length of the limbs 1721a, 1721b may also be selected such that the limbs follow a generally linear path or a path with only a slight curvature such that the don't bend back on themselves. This may result in easier pull-out of the limbs and/or a reduced likelihood of trauma to the wound during the removal process.

The first and second limbs 1721a, 1721b may be generally the same length, or one limb may be longer than the other. The connector 1718 may be shorter or longer than the limbs 1721a, 1721b. In embodiments in which the connector defines a portion of the conduit structure, the connector 1718 may be between about 10% and about 200% of the length of the limbs (or the length of the longer of the two limbs), preferably between about 60% and about 100%.

In alternative embodiments the connector joining two limbs of the conduit structure may be a non-resorbable component intended for removal from the wound along with the conduit structure at the conclusion of treatment. In such an embodiment one limb of the conduit structure would be permanently fixed to the connector, for example overmoulded with the connector, and the other limb would be removably attached to the connector such that the connector detached from that limb when the conduit structure was pulled out from the wound site.

For all embodiments, the devices may be engineered to provide a longer or shorter resorption time by adding additional layers of bioresorbable material to the sheath of the device, or by providing sheath layers that will be more quickly resorbed. Longer resorption time may be advantageous for sites which require prolonged periods of vacuum pressure, drainage or the delivery and removal of fluid. A prolonged ‘service lifetime’ of the device may also be obtained through the use of either non-absorbable suture material in truss-based conduit structures or long-lasting absorbable materials for the stitching on the seam features. The size of the device apertures can also be reduced in those situations where prolonged removal of fluid is favoured over the application of vacuum pressure to the surrounding tissue.

The device 1 . . . 1701 described herein is configured to allow the effective supply of fluid to and removal of fluid from a treatment site. Specifically, the fluid being supplied to and removed from the treatment site that is receiving a consistent vacuum pressure of between 60 mmHg to 150 mm Hg.

The treatment site may be a space between surfaces of muscle tissue, connective tissue or skin tissue that have been separated during surgery or as a result of trauma and/or any site where soft tissue has been removed or repaired. The device may also be wholly contained within a layer of tissue, such as the sub-cutaneous layer or muscle layer, where the application of vacuum pressure and/or the delivery and removal of fluid is desired. Some examples include the abdominal wall after surgery, or the breast post-mastectomy or breast reconstruction. The treatment site may be the site of a seroma or hematoma, or maybe used as a prophylactic following surgical excision of tissue. Alternatively, the treatment site may be an open wound such as following trauma, injury or surgical excision of necrotic or infected tissue which can either be closed via an advancement of a tissue flap or sealed using an occlusive layer, such as a drape, to ensure a level of vacuum pressure can be sustained.

The treatment site may also be a site traversing across one or more layers of tissue, for example, across all or a portion of the subcutaneous tissue layer, from the interface with the underlying muscle layer to the connection with the dermal or epidermal layer of skin. One example may be a treatment site at which the flange of the device was anchored or affixed to a muscle fascia at one side, with the remaining device positioned within the subcutaneous layers of tissue during closure of a primary surgical incision, such as following a caesarean incision or a laparotomy.

The tabs or flanges 7 . . . 1707 of the device 1 . . . 1701 advantageously allow the device to be secured at the wound site by suturing the tabs or flanges to tissue in a position where the application of vacuum pressure, fluid removal, and/or targeted delivery of treatment fluids is most desired. This allows the targeted administration of vacuum pressure to areas of the treatment site that would most benefit from the obliteration of post-surgical dead space and removal of fluid, such as a site with extensive resection where a resultant tissue gape or mismatch will exist following primary closure of the surgical site, for example an internal tumour site or a donor site.

The ability to retain the device in place at the treatment site also helps to ensure the device will continue to function for a prolonged period of time once the patient starts to move. In other prior art devices, unwanted movement of the device within the treatment site can cause further internal trauma, prevent the previously separated tissue planes from being held back together by any administered vacuum, and can cause the conduit to move to a site where the movement of bone and/or muscle could cause the conduit to become blocked or pinched within the body.

A suitably shaped device may be selected, or for some embodiments, the length or shape of the device may be adjusted to best suit the wound site and the desired treatment areas. This may also include selecting or adjusting or shaping the device to avoid proximity to area where the application of negative pressure may be undesirable, for example, where it may be unsafe. Such sites may include areas of ligated vessels, exposed nerves, or other sensitive vessels.

The device 1 . . . 1701 is used as part of a system for delivering and draining fluid from the treatment site. The device conduit structure holds the two tissue surfaces spaced apart, thereby defining a channel into which fluid from the treatment site can drain or from which fluid can be delivered to the treatment site. The two tissue surfaces need to be held apart because they would otherwise collapse together, particularly under application of negative or reduced pressure (vacuum) to assist with fluid drainage.

A port in the form of an opening or a pair of openings at one end of the device 1 . . . 1401, allows for connection of the channel with a source of negative pressure or positive pressure. The port may be a dual lumen conduit and/or may be provided by the exposed open ends of the supply and removal conduits 13 . . . 1713, 15, . . . 1716. A fluid supply conduit is releasably coupled to the port to be in fluid communication with the fluid supply lumen, and a fluid removal conduit is coupled to be in fluid communication with the fluid removal lumen.

In some embodiments, the port may be coupled to an impermeable dressing located on the exterior surface of the patient's skin which provides an airtight hermetic seal around the incision of the skin and an alternative means to which a conduit is releasably coupled to the dressing. In other embodiments, the port could be provided by a connector that interfaces with the conduit structure and an external device.

A reservoir is located external to the body of the patient, and arranged in fluid communication with the fluid removal lumen for receiving fluid from the device. The source of pressure may be capable of delivering negative pressure to the device so that fluid is drained from the treatment site into the device and transferred through the conduit to the reservoir and/or so that a treatment fluid is drawn through the conduit into the device and delivered to the treatment site, or may be capable of delivering positive pressure to the device so that fluid in the reservoir is transferred through the conduit into the device and to the treatment site. The treatment fluid may be a liquid or a gas, for example may include filtered air or other mixed phase fluids such as vapour or humidified air. In embodiments where a treatment liquid is introduced, a further reservoir containing or holding a treatment fluid may be coupled to the fluid supply lumen for delivering the treatment fluid to the device.

The source of pressure will typically be a pump for pumping fluid from the reservoir into the device for delivery to the treatment site or a vacuum pump for applying negative pressure to drain fluid from the treatment site. The system operates to substantially maintain the negative pressure of the treatment site during the introduction of filtered air and/or other treatment fluids. The pump may be manually operated, for example using a squeeze bulb, or may be electronically controlled for more precise delivery of fluid to the site. One particularly suitable pump is described in U.S. application No. 63/117,995, incorporated herein by reference.

In a system where fluid is being delivered to the treatment site, the fluid to be delivered may contain one or more nutrients, ‘flowable fluids’ such as Thixotropic gels or highly viscous fluids that can still be transported via a conduit, cell-suspensions therapeutic agents for promoting wound healing. The fluid may comprise flowable gels derived from ECM, hyaluronic acid, growth factors to aid healing, to antimicrobial drugs for the treatment of infection, analgesic drugs such as fentanyl or morphine for pain relief and anti-inflammatory drugs such as ketorolac or diclofenac, for example, although other fluids are envisaged and will be apparent to a skilled person.

In some alternative embodiments the device could be operably connected to one or more other devices, implanted at different respective sites for treating the respective sites with the same pressure source.

### Animal Studies

### Test 1

Prior to commencement of the animal study, a bench test was carried out in which the device 1101 illustrated in FIGS. 41 to 45 was placed within a sealed polyurethane bag and connected to the pump device described in our U.S. application No. 63/117,995. Blood from a blood bag was mixed with a coagulant to simulate a slow clot and instilled into the device 1101 to assess the ability of the implant to maintain the vacuum pressure at a treatment site when the clotted fluid is being removed by the connected pump via the repeated delivery of filtered air to the implant.

The test setup confirmed that an implant device such as the device 1101 of FIGS. 41 to 45, without sheath apertures, can effectively deliver the vacuum pressure to the treatment site.

The internal conduit structure of this implant was approximately 260 mm long and constructed using two Ø3.2 mm mandrels where a Ø450 μm (410-450 μm) monofilament polypropylene suture was used to construct a truss with a pitch length of 2.5 mm in between the nodes which proves an internal lumen area of ˜ 16 mm². The device 1111 was sewn with two runs of Ø125 μm (100-149 μm) PGA multifilament stitching along the seam 1109 on both sides of conduit structure to secure the upper and lower sheath sheets 1103a, 1103b over the conduit structure 1111.

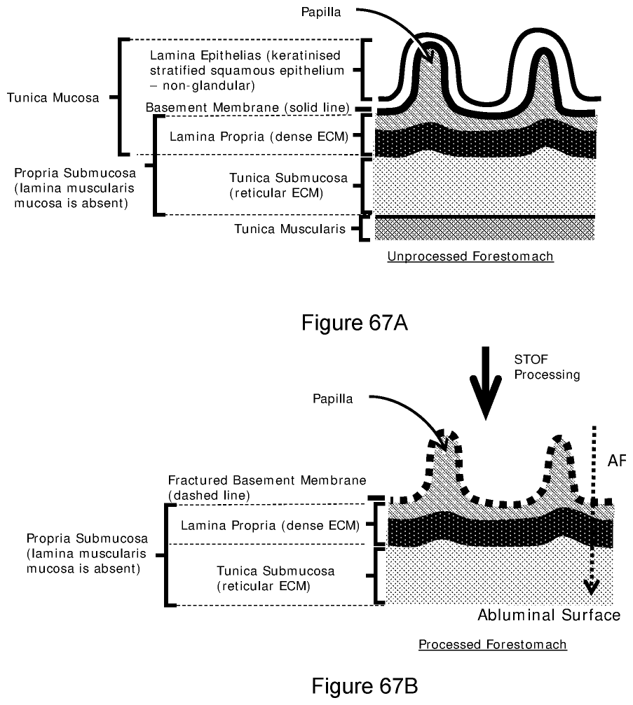

As illustrated in FIGS. 67A and 67B, fluid flow rates through ECM sheets is higher in one direction compared to the other direction. In embodiments tested, the ECM sheets were arranged with the papilla (luminal) surface of the ECM facing outward, away from the internal conduit structure 1111.

The device 1101 was placed into a sheep weighing 90 kg where the entire latissimus dorsi muscle was removed (weighing 195 g). The conduit structure 1111 of the device was coupled to a dual lumen silicone tube which had a fluid supply lumen size of 1.65 mm² (Ø1.45 mm) and a fluid removal lumen size of ˜ 9 mm² (equivalent diameter of Ø3.39 mm) via a push fit connector, which had an internal conduit area of 9.65 mm².

The multi-lumen silicone tube was in turn connected to an external battery powered vacuum device which was targeting the maintenance of the vacuum pressure measured along the fluid supply lumen of between 60 mmHg-115 mmHg, where the pump was configured to limit the vacuum being supplied along the fluid removal lumen to a maximum of 150 mmHg. The pump was configured to ensure that this level of vacuum pressure was maintained during the introduction of filtered ambient air which was controlled via a valve that was programmed on a cycled of 14 seconds open and 20 seconds closed. The valve cycle was operating in a continuous cycle until the pressure measured at along the fluid supply lumen reached a 60 mm Hg vacuum pressure threshold, at which point the system ceases to operate the valve.

The vacuum pressure measured at far end of the implant/along the fluid supply lumen of the tube was found to sustain the target 60-115 mmHg vacuum pressure for a period of approximately 5½ days following surgery where a total amount of 349 g of exudate was removed during this time.

Upon euthanasia of the study animal it was found that a large seroma had formed over the top of the implant device, concluding that while the device was effective for removing fluid for the entire duration of treatment it was not effective at managing the post-surgical dead space created by the complete resection of the latissimus dorsi muscle.

### Test 2

The animal study described above for test 1 above was repeated using an implant device 1201 shown in FIGS. 46 to 51, with the 00.5 mm aperture features in the sheath. In this test the only other variable modified was the addition of sterile saline supplied via the fluid supply lumen at periodic intervals over the first 3 days of treatment post-surgery, to assist in purging the device 1201 of any clotting factors such as fibrin/fibrinogen or other blood components. The sterile saline was delivered to the treatment site using a manual means to draw sterile saline through the implant by utilising the vacuum pressure being maintained within the implant device, which maintains the vacuum pressure at the treatment site throughout the introduction of the saline.

In this test a total of 345 g of saline was added, comprising of 92 g on the day following surgery (t=0), 142 g two days following surgery (t=2) and 111 g three days (t=3) following surgery. The vacuum pressure measured along the fluid supply lumen was found to fall below the 60 mm Hg lower vacuum pressure threshold at approximately 2½ days following surgery at which point the opening and closing of the air valve ceases to function, with the system targeting a constant vacuum pressure level within the system at a single pressure level.

A sheep weighing 87 kg was used for the study with the removed latissimus dorsi muscle weighing 116 grams. A total of 800 grams of exudate was removed from the animal over a period of 5 days following surgery.

Upon euthanasia of the study animal it was found that a clearly defined line of highly opposed tissue crossing over the middle of the device with approximately 50-70% of the device well integrated with the surrounding tissue. The region of the defect site laying in closer proximity to the ulna (cranial end of the animal), accounting for approximately 30-50% of the device, was found to have a seroma, with the opposing side of the defect area extending towards the rear (caudal end of the animal) appearing to be completely healed with well opposed tissue.

The gross observations from this euthanasia found there to be significantly improved clinical outcomes for dead space management following treatment when compared to the previous device.

### Test 3

A further animal study was performed to compare the treatment outcomes of a linear device similar to the device 101 shown in FIGS. 16-21 to an existing prior art closed wound drainage device (control device).

The closed wound drainage (control) device was a Cardinal Health 3-spring mechanically powered closed wound drainage device with a perforated 15 Fr (Ø5 mm O.D tube-Ø3 mm ID tube) sized PVC drain (part number SU130-403D). These reservoirs are known to deliver approximately 70 mmHg of vacuum pressure when fully primed.

The treatment device was approximately ˜ 100 mm in length where the central conduit device was a multi-lumen silicone tube with a cross sectional shape shown in FIG. 21. The fluid supply lumen 113 is Ø1.4 mm and the fluid removal lumen 111 is approximately 18 mm² in area. The conduit was fabricated as shown in the embodiment in FIGS. 28 and 29 with a series of 3x Ø3 mm sized cut apertures 106 positioned around the top, side, and bottom face of the fluid removal lumen, which are distributed along the length of the conduit that is positioned within the sleeve. The internal wall between the two lumens 113, 115 was back cut in the manner illustrated in FIGS. 28 and 29 to ensure the path between the fluid supply lumen and the opening of the fluid removal lumen is preserved when the device is under vacuum.

The implant device was fabricated with two sheets of ECM material which were sewn using Ø125 μm (100-149 μm) PGA multifilament stitching along the seam on both sides to secure the sleeve with the papilla (luminal surface) side of the device facing inwards towards the conduit. Both sides of the sleeve device contained a series of Ø0.5 mm diameter apertures along the length of the device.

The external vacuum pump device connected to this implant was configured to open the fluid supply valve for 14 Seconds with a closed duration of 2 minutes which introduces filtered air into the conduit via the fluid supply lumen with the system maintained at a vacuum pressure level of 80 mmHg during the instillation of filtered air. Once the air valve closes to return the device to a second equilibrium pressure of 100 mmHg. This cycle continues to operate until the point at which the pressure being measured along the fluid supply lumen of the device drops to 60 mmHg or below, at which point the opening cycle of the valve ceases to operate and the system targets the constant delivery of 100 mm Hg along the fluid removal lumen of the device. The system is programmed to ensure the vacuum pressure level along the fluid removal conduit does not exceed 150 mmHg as a safety mechanism.

An ovine bi-lateral external abdominal oblique dead space seroma model was used to compare the two devices. In this study a sheep weighing 58.5 kg was used with one side receiving the treatment device and the other receiving the closed wound drainage (control) device.

The closed wound drainage device defect site was created by excising 18 grams of external abdominal oblique muscle from an undermined area above the muscle to create a resultant defect area of approximately 38 cm². The drainage catheter was placed into the wound at the lowermost aspect of the wound with the tube also ported at the lowermost aspect of the wound. The tube was routed alongside the external surface of the sheep and was connected to a spring reservoir located within an equipment harness located on the back of the animal, which provides the vacuum pressure to the catheter. The reservoir was primed and connected to the treatment device once the wound was closed to administer the 70 mmHg of vacuum pressure to the perforated catheter.

The treatment device was placed within a defect that was created on the opposing side of the animal. A defect site of ˜ 82 cm² in area was created by excising 19.5 grams of external abdominal oblique muscle from an undermined area above the muscle. The implant device was positioned along the longitudinal axis of the defect site which extended vertically across a defect site on an angle. The implant device was secured to the treatment site using a series of passed sutures that were tied off to affix the implant in place. Once the treatment site was closed the implant device was connected to the externally mounted vacuum pump device to function as programmed.

For this animal study the treatment duration for both the treatment and control devices were set to run for 14 days following surgery. The manually powered closed wound drainage (control) device was primed each day to ensure the continual application of negative pressure where the amount of wound exudate collected by both devices was also weighed and recorded each day.

An ultrasound assessment was performed at days 7 and 14 post-surgery to assess the size of any seroma forming at the defect site for both devices. The volume of any seroma measured at the defect site was calculated using the formula to determine the volume of an ellipsoid.

Both the treatment and control devices were removed at 14 day post-surgery using a force gauge to determine the removal force. The animal was euthanised at 28 day post-surgery to perform a gross assessment of the defect site for both animals.

The results from the study are shown in the table below;

| Total Collected | | Seroma Volume | | Seroma Volume | | MAX Tube | |
|---|---|---|---|---|---|---|---|
| Fluid after 7 | | on Day 7 using | | on Day 14 using | | Removal | |
| Days (mL) | | Ultrasound (mL) | | Ultrasound (mL) | | Force (N) | |
| Treatment | Control | Treatment | Control | Treatment | Control | Treatment | Control |
| 153 | 55 | 0 | 0 | 0 | 521 | 5.46 | 8.06 |

Following 7 days of treatment the closed wound drainage (control) device was found to collect a total of 55 mL's of wound exudate where the treatment device was found to collect 153 mL's of exudate. There was no seroma present at either the 7 day or 14-day post-surgery timepoint for the treatment device, with the closed wound drainage (control) device found to have zero seroma at the 7-day time point and a large ˜521 mL (cm³) seroma at the defect set following 14 days post-surgery.

The force required to remove the conduit of the treatment device from the animal at the 14-day post-surgery treatment end point was recorded at a maximum of 5.5 N with the PVC tube from the closed wound drainage (control) device requiring a maximum of 8.1 N.

A gross examination of the defect sites for both the treatment and closed wound drainage (control) devices was performed at 28 days post-surgery. The defect site with the treatment device was found to be completely integrated with no signs of any seroma or wound fluid at the defect site. The defect site with the closed wound drainage (control) device was found to have a large seroma consistent with the ultrasound findings at the 14-day post-surgical timepoint, with virtually zero signs of any integration of the separated tissue planes of the defect site.

The results from this observation confirmed that the treatment device provided complete dead space closure of the defect site following surgery.

Although the invention has been described by way of example, it should be appreciated that variations and modifications may be made without departing from the scope of the invention as defined in the claims. The embodiments described herein are provided to exemplify alternatives for various features of the device. It will be appreciated that many permutations of these features are possible to create other embodiments within the scope of the invention claimed. That is, features from one embodiment may be combined with features of another embodiment to create a new embodiment that remains within the scope of the present invention. Furthermore, where known equivalents exist to specific features, such equivalents are incorporated as if specifically referred in this specification.

The device described herein may advantageously be customised to adjust the duration for which the device is functional in-situ for any given application. For example, by adjusting channel size, wall thicknesses, or the thickness or density of truss members, or the number and type of bracing members.

1. A device for implantation at a treatment site in the body of a patient for the removal of fluid from the treatment site; the device comprising:
a conduit structure at least in part defining a fluid removal lumen, the conduit structure comprising a first removable piece and a second removable piece;
a connector removably and coaxially coupling the first piece of the conduit structure to the second piece of the conduit structure; and
a porous bioresorbable sheath surrounding at least a portion of the conduit structure;
wherein the connector is configured to decouple from one or both of the conduit pieces upon initiation of removal of the conduit structure or a part thereof from the treatment site at the completion of treatment.

2. A device as claimed in claim 1, wherein the device comprises an inlet port and an outlet port and the conduit structure extends between the inlet port and the outlet port.

3. A device as claimed in claim 1, comprising a dual lumen port for connection with one or more external components, wherein a first lumen of the port is connected to a first piece of the conduit structure and a second lumen of the port is connected to a second piece of the conduit structure.

4. A device as claimed in claim 2 or 3, wherein the connector is at a position on the device distal to the port(s).

5. A device as claimed in any preceding claim, wherein the two pieces of the conduit structure have abutted ends.

6. A device as claimed in any preceding claim, wherein the two pieces of the conduit structure are substantially the same length.

7. A device as claimed in any preceding claim, wherein the connector is tubular.

8. A device as claimed in any preceding claim, wherein a first end of the connector is configured to fit snuggly in a lumen of the first part of the conduit structure and a second end of the connector is configured to fit snuggly in a lumen of the first part of the conduit structure.

9. A device as claimed in any preceding claim wherein the connector is a bioresorbable component.

10. A device as claimed in any preceding claim, wherein the connector comprises a bioresorbable resilient truss having one or more flexible elongate wall members wound in a manner to define a channel, and one or more bracing members linked to the elongate wall member(s).

11. A device as claimed in 10, comprising two flexible elongate wall members wound in a manner to define a channel, the two elongate wall members intersecting each other periodically at a plurality of cross-over nodes.

12. A device as claimed in claim 11, wherein the two wall members are oppositely wound.

13. A device as claimed in 11, wherein a first one of the wall members is a left-side wall member, and a second one of the wall members is a right-side wall member.

14. A device as any one of claims 11 to 13, comprising at least two flexible elongate bracing members, each bracing member being linked to the two elongate wall members at a plurality of the cross-over nodes.

15. A device as claimed in 14, wherein the bracing members are provided along the top and bottom of the channel.

16. A device as claimed in any one of claims 10 to 15, wherein each bracing member is mechanically linked to the two elongate wall members at the respective cross-over nodes by way of the respective bracing member looping around the wall members.

17. A device as claimed in any preceding claim, wherein the connector is attached to the sheath.

18. A device as claimed in any preceding claim, wherein the bioresorbable sheath comprises a plurality of apertures positioned to enable fluid communication between the treatment site and the conduit structure, the apertures each having an area of about 1 mm² or less.

19. A device as claimed in any preceding claim, wherein the sheath comprises one or more top sheets that extends over a top part of the conduit structure, and one or more bottom sheets that extend over a bottom part of the conduit structure.

20. A device as claimed in claim 19, wherein the top and bottom sheets are stitched together.

21. A device as claimed in claim 20, where in the connector is tied to a row of stitching.

22. A device as claimed in any one of claims 19 to 21, wherein the top and bottom sheets are mechanically interlocked together.

23. A device as claimed in claim 22, wherein the sheath comprises a first sheet having a plurality of lugs and a second sheet having a plurality of apertures, each lug of the first sheet being located through a respective aperture in the second sheet to interlock the first sheet with the second sheet.

24. A device as claimed in claim 23, wherein the top sheet comprises a plurality of lugs and the underlying sheet(s) comprise(s) a plurality of apertures, each lug of the top sheet being located through a respective aperture in the underlying sheet(s) to interlock the sheets of the sheath.

25. A device as claimed in claim 24, wherein the holes and the lugs are dimensioned so that the lugs engage with a surface of the second sheet.

26. A device as claimed in any preceding claim, wherein the sheath comprises an end section proximal an inlet and outlet of the device, configured to prevent or minimise the ingress of wound debris into the conduit structure.

27. A device as claimed in claim 26, wherein the end section of the sheath does not comprise through apertures.

28. A device as claimed in any preceding claim, wherein the sheath comprises one or more layers of extracellular matrix (ECM) or polymeric material.

29. A device as claimed in claim 28, wherein the ECM is formed from decellularised propria-submucosa of a ruminant forestomach.

30. A device as claimed in any one of claims 1 to 29, wherein the removable conduit structure comprises a silicone form.

31. A device as claimed in any preceding claim, wherein the fluid removal lumen has a cross-sectional area of at least 7 mm².

32. A device as claimed in claim 31, wherein the fluid removal lumen has a cross-sectional area of about 18 mm².

33. A device as claimed in any preceding claim, wherein the sheath comprises a sealing end section free from apertures and having a tight fit with the underlying portion of the conduit structure.

34. A device as claimed in claim 33, wherein the sealing end section of the sheath extends over a portion of the conduit structure that comprises fluid impervious walls.

35. A device as claimed in any preceding claim, wherein the connector is an elongate component that forms a portion of the conduit structure and which defines a respective portion of the fluid removal lumen.

36. A system for draining fluid from a treatment site and delivering fluid to a treatment site in the body of a patient comprising:
(xi) a device as claimed in any one of claims 1 to 35;
(xii) a conduit releasably coupled to either a port of the device or to a fluid impermeable dressing;
(xiii) a reservoir located external to the body of the patient and containing a treatment fluid, the reservoir in fluid communication with the fluid supply lumen;
(xiv) a second reservoir located external to the body of the patient, the second reservoir in fluid communication with fluid removal lumen for receiving fluid from the device; and
(xv) a source of pressure coupled to the conduit for delivering positive pressure or negative pressure to the device.

37. A system as claimed in claim 36, wherein the source of pressure is capable of delivering negative pressure to the device so that fluid is drained from the treatment site into the device and transferred through the conduit to the reservoir.

38. A system as claimed in claim 36 or 37, wherein the port of the device is positioned external to the patient's body.

39. A kit of parts for forming the device as claimed in any one of claims 1 to 35, comprising a two-piece conduit structure defining a fluid removal lumen, a connector for removably and coaxially coupling a first piece of the conduit structure to a second piece of the conduit structure; and a bioresorbable sheath defining a passage for receipt of the conduit structure.

40. A kit of parts as claimed in claim 39, wherein the bioresorbable sheath is generally tubular having two open ends.
